(19) United States
(12) Patent Application Publication
(10) Pub. No.: US 2025/0099241 A1
(43) Pub. Date: Mar. 27, 2025

(54) DELIVERY SYSTEMS FOR CARDIAC VALVE REPAIR DEVICES, AND ASSOCIATED METHODS OF OPERATION

(71) Applicant: Half Moon Medical, Inc., Menlo Park, CA (US)

(72) Inventors: Neil Zimmerman, Menlo Park, CA (US); Brian Y. Tachibana, Danville, CA (US); Matthew McLean, III, San Francisco, CA (US); Robert O’Grady, San Francisco, CA (US); Jose Gonzalez, Fremont, CA (US); Erik Thai, San Jose, CA (US)

(21) Appl. No.: 18/897,448

(22) Filed: Sep. 26, 2024

Related U.S. Application Data
(60) Provisional application No. 63/585,755, filed on Sep. 27, 2023.

(51) Int. Cl. A61F 2/24

(52) U.S. Cl. CPC A61F 2/243; A61F 2220/0008

(57) ABSTRACT

Delivery systems for implanting cardiac valve repair devices are disclosed herein. In some embodiments, a delivery system for implanting a valve repair device includes a delivery catheter, an implant shaft, a drive shaft, a clip cable, and a cinching line. The delivery catheter can hold the device in a compressed configuration. The implant shaft can have a hub assembly releasably coupled to a portion of the device, and the drive shaft can be actuated to release the portion of the device from the hub assembly. The clip cable can be releasably coupled to a clip of the device for opening and closing the clip to capture and secure a portion of a native leaflet. The cinching line can extend around a portion of the device for cinching the device to a compressed position when deployed from within the delivery catheter.

### CROSS-REFERENCE TO RELATED APPLICATION

This application claims the benefit of U.S. Provisional Patent Application No. 63/585,755, filed Sep. 27, 2023, and titled “DELIVERY SYSTEMS FOR CARDIAC VALVE REPAIR DEVICES, AND ASSOCIATED METHODS OF OPERATION,” which is incorporated herein by reference in its entirety.

### TECHNICAL FIELD

The present technology generally relates to delivery systems for implanting cardiac valve repair devices via a minimally invasive procedure, such as an endovascular approach.

### BACKGROUND

Proper functioning of the mitral valve can be affected by mitral valve regurgitation, mitral valve prolapse, and/or mitral valve stenosis. Mitral valve regurgitation can occur when the leaflets of the mitral valve fail to coapt into apposition at peak contraction pressures such that blood leaks from the left ventricle into the left atrium. Several structural factors may affect the proper closure of the mitral valve leaflets. For example, an enlarged mitral annulus caused by dilation of heart muscle may prevent proper coaptation of the leaflets during systole. Other conditions involve a stretch or tear in the chordae tendineae—the tendons connecting the papillary muscles to the inferior side of the mitral valve leaflets-which may also affect proper closure of the mitral annulus. A ruptured chordae tendineae, for example, may cause a valve leaflet to prolapse into the left atrium due to inadequate tension on the leaflet. Abnormal backflow can also occur when the papillary muscles are compromised (e.g., due to ischemia) such that the affected papillary muscles do not contract sufficiently to effect proper closure during systole.

Mitral valve prolapse can occur when the mitral leaflets abnormally bulge up into the left atrium, which can also lead to mitral valve regurgitation. Normal functioning of the mitral valve may also be affected by mitral valve stenosis, or a narrowing of the mitral valve orifice, which impedes of filling of the left ventricle during diastole.

Mitral valve regurgitation is often treated using diuretics and/or vasodilators to reduce the amount of blood flowing back into the left atrium. Other treatment methods, such as surgical approaches (open and intravascular), have also been used to either repair or replace the native mitral valve. For example, cinching or resecting portions of the dilated annulus are typical repair approaches. Cinching of the annulus has been accomplished by implanting annular or peri-annular rings which are generally secured to the annulus or surrounding tissue. Other repair procedures have also involved suturing or clipping of the valve leaflets into partial apposition with one another. Alternatively, more invasive procedures replace the entire valve with mechanical valves or biological tissue. These invasive procedures are conventionally done through large open thoracotomies and are thus very painful, have significant morbidity, and require long recovery periods.

However, with many repair and replacement procedures, the durability of the devices or improper sizing of annuloplasty rings or replacement valves may cause complications. Moreover, many of the repair procedures depend upon the skill of the cardiac surgeon since poorly or inaccurately placed sutures may affect the success of procedures.

Compared to other cardiac valves, the mitral valve presents unique challenges because portions of the mitral valve annulus have limited radial support from surrounding tissue and the mitral valve has an irregular, unpredictable shape. For example, the anterior wall of the mitral valve is bound by only a thin wall separating the mitral valve annulus from the inferior portion of the aortic outflow tract. As a result, significant radial forces on the mitral valve annulus are not acceptable as they could lead to collapse of the inferior portion of the aortic tract with potentially fatal consequences. Another challenge of the mitral valve anatomy is that the maze of chordae tendineae in the left ventricle makes navigating and positioning a deployment catheter much more difficult compared to other heart valves. Given the difficulties associated with current procedures, there remains the need for simple, effective, and less invasive devices and methods for treating dysfunctional heart valves. Additionally, since it is also difficult to deliver devices to the mitral valve, there also remains the need for effective and less invasive delivery systems to deliver the implantable cardiac devices to the mitral valve.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIG. 1 is a diagram of a mitral valve that may be accessed by a delivery system in accordance with embodiments of the present technology.

FIG. 2 is a perspective side view of a delivery system configured in accordance with embodiments of the present technology.

FIGS. 3A and 3C are enlarged isometric side views of a distal portion of the delivery system of FIG. 2 with an implantable device in an uncinched position and a cinched position, respectively, in accordance with embodiments of the present technology. FIG. 3B is a top view of the implantable device of FIG. 2 in a deployed position in accordance with embodiments of the present technology.

FIGS. 4A and 4B are enlarged side views of a distal portion of an implant shaft of the delivery system of FIG. 2 including a hub assembly in accordance with embodiments of the present technology.

FIGS. 5A and 5B are isometric side views of an inner hub component of the hub assembly of FIGS. 4A and 4B in a retracted position in accordance with embodiments of the present technology. FIG. 5C is an isometric side view of the inner hub component of FIGS. 4A and 4B in an extended position in accordance with embodiments of the present technology.

FIGS. 6A and 6B are a side view and a side cross sectional view, respectively, of a clip assembly of the implantable device of FIGS. 3A and 3B coupled to a distal portion of a clip cable assembly of the delivery system of FIG. 3A in a locked position in accordance with embodiments of the present technology. FIGS. 6C and 6D are perspective side views of the clip cable assembly of FIG. 3A in a locked position and an unlocked position, respectively, in accordance with embodiments of the present technology. FIG. 6E is a cross-sectional side view of the clip assembly of the implantable device of FIGS. 3A and 3B decoupled from the clip cable assembly of FIG. 3A in an unlocked position in accordance with embodiments of the present technology.

FIGS. 7A and 7B are a top view and a side view, respectively, of an implant handle of the delivery system of FIG. 2 in accordance with embodiments of the present technology.

FIG. 8 is a partially cross-sectional isometric view of a connector and an atrial-fixation release actuator of the implant handle of FIGS. 7A and 7B in accordance with embodiments of the present technology.

FIG. 9 is a perspective top view of the implant handle of FIGS. 7A and 7B in accordance with embodiments of the present technology.

FIG. 10A is a partially-transparent isometric view of a cinch mount assembly of the implant handle of FIG. 9 in accordance with embodiments of the present technology. FIGS. 10B and 10C are perspective side views of the cinch mount assembly of FIG. 10A in accordance with embodiments of the present technology.

FIG. 11A is an enlarged isometric view of a clip cable mount assembly and a portion of a rotatable sleeve of the implant handle of FIG. 9 in accordance with embodiments of the present technology. FIG. 11B is a cross-sectional side view of a telescoping assembly, rotatable sleeve, and a clip actuator of the implant handle of FIG. 9 in accordance with embodiments of the present technology. FIG. 11C is an enlarged top view of the clip cable mount assembly of FIG. 9 in accordance with embodiments of the present technology. FIGS. 11D and 11E are enlarged top views of the clip cable mount assembly of FIG. 9 in accordance with embodiments of the present technology.

FIGS. 12A and 12B are a proximally-facing isometric view and a cross-sectional side view, respectively, of the handle of FIGS. 7A and 7B including a cinch actuator, a first leadscrew, and a second leadscrew in accordance with embodiments of the present technology.

FIGS. 13A-13C are perspective top views of the implant handle of FIGS. 7A and 7B in a cinched position, a partially-cinched position, and an uncinched position, respectively, in accordance with embodiments of the present technology.

FIG. 14 is a cross-sectional side view of a lockwire release assembly of the implant handle of FIGS. 7A and 7B in accordance with embodiments of the present technology.

FIG. 15 is an isometric view of a proximal portion of the implant handle of FIGS. 7A and 7B including a clip position indicator assembly in accordance with embodiments of the present technology.

FIGS. 16A-16N illustrate different top and/or side views of the implantable device and the distal portion of the delivery system of FIG. 2 during a procedure to implant the implantable device at a native mitral heart valve of a patient in accordance with embodiments of the present technology.

FIGS. 17A and 17B are perspective side views of a clip cable assembly in a locked position and an unlocked position, respectively, relative to a clip assembly of the implant of FIGS. 3A and 3B in accordance with additional embodiments of the present technology. FIG. 17C is a partially-transparent side view of the clip cable assembly and the clip assembly of FIGS. 17A and 17B in the locked position in accordance with embodiments of the present technology.

FIGS. 18A-18C are enlarged side views of a distal portion of the implant shaft of the delivery system of FIG. 2 during actuation of a clip release actuator of FIG. 14 in accordance with embodiments of the present technology.

FIG. 19 is a perspective view of a portion of the implant handle of FIGS. 7A and 7B in accordance with additional embodiments of the present technology.

### DETAILED DESCRIPTION

Aspects of the present disclosure are directed generally to delivery systems for implanting a medical device, such as a cardiac valve repair device, in a subject (e.g., a human patient). In several of the embodiments described below, the implantable medical device can include a coaptation member, a clip assembly coupled to the coaptation member, and a fixation member extending from the coaptation member. The coaptation member can be configured to (i) displace at least a portion of a first native leaflet of a heart valve of the subject and (ii) coapt with at least a portion of a second native leaflet of the heart valve. The fixation member can contact tissue of the heart above the heart valve (e.g., atrial tissue) to anchor the coaptation member in position. The clip assembly can secure at least a portion of the first native leaflet to the coaptation member.

In several of the embodiments described below, a delivery system for delivering the implantable medical device to the heart valve includes a delivery catheter, an implant shaft extending through the delivery catheter, a drive shaft extending at least partially through the implant shaft, a clip cable extending through the implant shaft, and one or more cinching lines extending through the implant shaft. The delivery catheter can hold the implantable medical device in a compressed configuration via tension on the cinching line(s) which encircle the fixation member. The implant shaft can include a hub assembly releasably coupled to the fixation member for steering the implantable medical device to the heart valve. The drive shaft can be operably coupled to the hub assembly for releasing the atrial-fixation member. The clip cable can be releasably coupled to the clip assembly for opening and closing the clip assembly to, for example, capture and secure the portion of the first native leaflet. The cinching line(s) can extend around the atrial-fixation member for cinching the fixation member to a compressed position when deployed from within the delivery catheter.

In some embodiments, the delivery system further includes an implant handle operably coupled to the implant shaft, the drive shaft, the clip cable, and the cinching line(s). The implant handle can be moved distally and proximally to advance and retract, respectively, the implant shaft relative to the delivery catheter to, for example, deploy the implantable medical device from the delivery catheter. The implant handle can include a clip actuator operably coupled to the clip cable and actuatable to rotate the clip cable to open and close the clip assembly. The implant handle can further include a cinch actuator operably coupled to the cinching line(s) and actuatable to both (i) reduce a tension in the cinching line(s) to allow the atrial-fixation member to expand for placement and delivery and (ii) increase a tension in the cinching line(s) to cinch the atrial-fixation member to the compressed position for repositioning or recapture of the implantable medical device. The implant handle can further include an atrial-fixation release actuator operably coupled to the drive shaft and actuatable to rotate the drive shaft to actuate the hub assembly to release the atrial-fixation member.

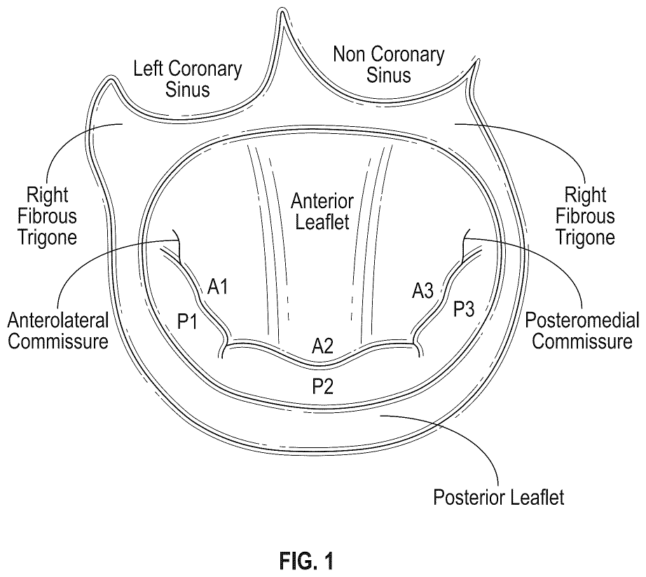

Specific details of several embodiments of the present technology are described herein with reference to FIGS. 1-19. The present technology, however, can be practiced without some of these specific details. In some instances, well-known structures and techniques often associated with catheter-based delivery systems, valve repair devices, delivery handles, steerable catheters, etc., have not been shown in detail so as not to obscure the present technology. The terminology used in the description presented below is intended to be interpreted in its broadest reasonable manner, even though it is being used in conjunction with a detailed description of certain specific embodiments of the disclosure. Certain terms can even be emphasized below; however, any terminology intended to be interpreted in any restricted manner will be overtly and specifically defined as such in this Detailed Description section.

The accompanying Figures depict embodiments of the present technology and are not intended to be limiting of its scope. The sizes of various depicted elements are not necessarily drawn to scale, and these various elements can be arbitrarily enlarged to improve legibility. Component details can be abstracted in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the spirit or scope of the present technology.

With regard to the terms “distal” and “proximal” within this description, unless otherwise specified, the terms can reference a relative position of the portions of a valve repair device and/or an associated delivery device with respect to an operator and/or a location in the vasculature or heart. For example, in referring to a delivery catheter suitable to deliver and position various valve repair devices described herein, “proximal” can refer to a position closer to the operator of the device or an incision into the vasculature, and “distal” can refer to a position that is more distant from the operator of the device or further from the incision along the vasculature (e.g., the end of the catheter). With respect to a heart valve repair device, the terms “proximal” and “distal” can refer to portions of the device relative to the native annulus. For example, “proximal” can refer to an upstream portion of the device spaced apart from the native annulus, and “distal” can refer to a downstream position at or proximate to the native annulus.

Further, as used herein, the designations “forward,” “rearward,” “upward,” “downward,” “top,” “bottom,” etc., are not meant to limit the referenced component to use in a specific orientation. It will be appreciated that such designations refer to the orientation of the referenced component as illustrated in the Figures. However, the systems of the present technology can be used in any orientation suitable to the user.

FIG. 1 is a diagram of a mitral valve that may be accessed by a delivery system in accordance with embodiments of the present technology. The anterior leaflet has a semi-circular shape and attaches to approximately two-fifths of the annular circumference. The motion of the anterior leaflet defines an important boundary between the inflow (diastole) and outflow (systole) tracts of the left ventricle. The posterior leaflet of the mitral valve has a crescent shape and is attached to approximately three-fifths of the annular circumference. The posterior leaflet typically has two well-defined indentations which divide the leaflet into three individual scallops identified as P1 (lateral scallop), P2 (middle scallop), and P3 (medial scallop). The three corresponding segments of the anterior leaflet are identified as A1 (lateral segment), A2 (middle segment), and A3 (medial segment). The leaflet indentations aid in opening the posterior leaflet during diastole.

As shown in FIG. 1, the mitral valve has anterolateral and posteromedial commissures which define a distinct area where the anterior and posterior leaflets come together at their insertion into the annulus. Sometimes the commissures exist as well-defined leaflet segments, but often this area is a subtle structure that can be identified using the following two anatomic landmarks: (a) the axis of corresponding papillary muscles, and (b) the commissural chordae, which have a specific fan-like configuration. Several millimeters of valvular tissue separate the free edge of the commissures from the annulus.

The mitral valve is an atrio-ventricular valve separating the left atrium from the left ventricle. The mitral annulus constitutes the anatomical junction between the left ventricle and the left atrium. The fixed ends of the leaflets are attached to the annulus. The anterior portion of the mitral annulus is attached to the fibrous trigones and is generally more developed than the posterior annulus. The right fibrous trigone is a dense junctional area between the mitral valve, tricuspid valve, non-coronary cusp of the aortic valve, and the membranous septum. The left fibrous trigone is situated at the junction of both left fibrous borders of the aortic valve and the mitral valve.

The mitral annulus is less well developed at the insertion site of the posterior leaflet. This segment is not attached to any fibrous structures, and the fibrous skeleton in this region is discontinuous. This posterior portion of the annulus is prone to increase its circumference when mitral regurgitation occurs in association with left atrial or left ventricular dilation. The mitral annulus is saddle-shaped, and during systole the commissural areas move proximally—that is, towards the roof of the atrium—while annular contraction also narrows the circumference. Both processes aid in achieving leaflet coaptation, which may be adversely affected by annular dilatation and calcification. The mitral annulus is surrounded by several important anatomic structures, including the aortic valve, the coronary sinus, and the circumflex artery. As a result, implanted cardiac devices at the mitral valve need to be positioned to accommodate the asymmetrical anatomy of the mitral valve without impacting the surrounding cardiac structures.

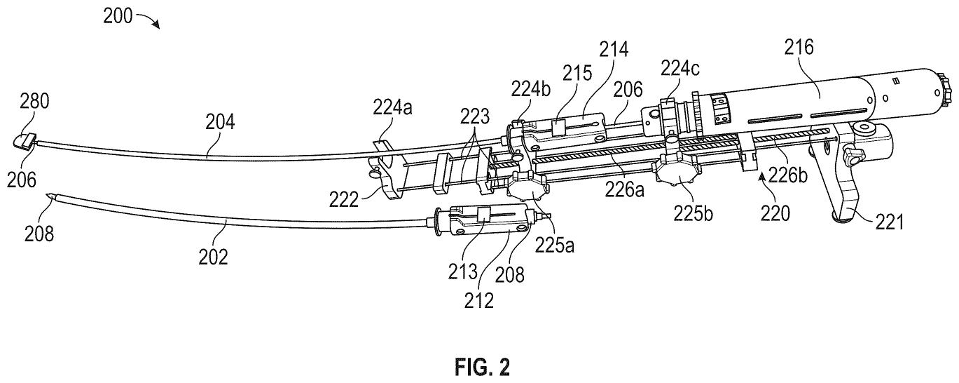

FIG. 2 is a perspective side view of a delivery system 200 configured in accordance with embodiments of the present technology. The delivery system 200 can be used to deliver an implantable device 280 to a heart of a subject (e.g., a human patient). The implantable device 280 can also be referred to herein as a “mitral valve repair device,” a “valve repair device,” a “coaptation assist device,” and/or the like. In some embodiments, the delivery system 200 can include some features that are at least generally similar in structure and function, or identical in structure and function, to corresponding features of one or more of the delivery systems described in U.S. patent application Ser. No. 17/194,113, titled “DELIVERY SYSTEMS FOR CARDIAC VALVE DEVICES, AND ASSOCIATED METHODS OF OPERATION,” and filed Mar. 5, 2021, which is incorporated herein by reference in their entirety.

In the illustrated embodiment, the delivery system 200 includes three nested/coaxial catheter/shaft structures: (i) an outer guide catheter 202, (ii) a delivery catheter 204 (also referred to as a “sleeve”) configured to extend at least partially through the guide catheter 202, and (iii) an implant shaft 206 (also referred to as a “brim shaft”) configured to extend at least partially through the delivery catheter 204 (collectively “catheters 202-206” or “shafts 202-206”). The catheters 202-206 can be individually manipulated and/or moved relative to one another to facilitate deployment of the implantable device 280. More specifically, in the illustrated embodiment (i) the guide catheter 202 is coupled (e.g., fixed) to a guide catheter handle 212 (also referred to as a “first handle 212”), (ii) the delivery catheter 204 is coupled to a delivery catheter handle 214 (also referred to as a “second handle 214”), and (iii) the implant shaft 206 is coupled to an implant handle 216 (also referred to as a “third handle 216” or a “deployment handle 216”); collectively “handles 212-216”). In some embodiments, two or more of the handles 212-216 can be integrated into a single handle assembly. In some embodiments, the delivery system 200 further includes a dilator assembly 208 configured to be advanced/retracted through the guide catheter 202 before introduction of the delivery catheter 204 and/or the implant shaft 206.

In some embodiments, the handles 212-216 and/or portions of the catheters 202-206 are coupled/mounted to a common handle support assembly 220 (also referred to as a “rack assembly” or “control rack”) that facilitates relative movement between the individual catheters 202-206, while maintaining the handles 212-216 in a stable, supported position and inhibiting unwanted movement therebetween. The handle support assembly 220 can include a proximal fixed portion 221 (e.g., a first stand), a distal fixed portion 222 (e.g., a second stand), and one or more tracks 223 extending at least partially between the proximal and distal fixed portions 221, 222.

The handle support assembly 220 can further include a plurality of mounts 224 (identified individually as first through third mounts 224-224c, respectively) coupled to one or more of the tracks 223. The first mount 224a can be coupled to or formed integrally with the distal fixed portion 222 of the handle support assembly 220 and is configured (e.g., shaped, sized) to receive and secure the guide catheter handle 212. The guide catheter handle 212 can be removably mounted to the first mount 224a, and is shown removed from the first mount 224a in FIG. 2. The second mount 224b is configured to receive and secure the delivery catheter handle 214, and includes a first actuation member 225a (e.g., a wheel, slider, knob, button) for individually moving the second mount 224b—and the delivery catheter handle 214 and the delivery catheter 204 coupled thereto—linearly/axially along the tracks 223 relative to the guide catheter handle 212 and/or the implant handle 216. For example, the first actuation member 225a can engage a first leadscrew 226a of the handle support assembly 220 for driving the delivery catheter handle 214 linearly along the first leadscrew 226a and the tracks 223. The third mount 224c is positioned proximal of the second mount 224b and is configured to receive and secure the implant handle 216. The third mount 224c similarly includes a second actuation member 225b for individually moving the third mount 224c and the implant handle 216 relative to the guide catheter handle 212 and/or the delivery catheter handle 214. For example, the second actuation member 225b can engage a second leadscrew 226b of the handle support assembly 220 for driving the implant handle 216 linearly along the second leadscrew 226b and the tracks 223.

The guide catheter 202 and the delivery catheter 204 can each have varying stiffnesses along a length thereof and/or can be steerable catheters that allow the guide and delivery catheters 202, 204 to deflect along one or more axes. In some embodiments, for example, the guide catheter handle 212 includes a guide actuation member 213 (e.g., a wheel, lever, knob, slider) that is actuatable to deflect a distal portion of the guide catheter 202. More specifically, the guide actuation member 213 can be coupled to a pull wire that is attached to a pull ring fixed at a distal portion of the guide catheter 202. Similarly, the delivery catheter handle 214 can include a delivery actuation member 215 that is actuatable to deflect a distal portion of the delivery catheter 204. In some embodiments, the guide catheter 202 has a diameter of less than about 30 French (e.g., about 29.5 French or less) and the delivery catheter 204 has a diameter of about 26 French or less.

During a delivery procedure using the delivery system 200, the distal portion of the delivery catheter 204 retains the implantable device 280 in a compressed delivery state and, upon reaching a target region (e.g., in the left atrium), begins to deploy (e.g., unsheathe) the implantable device 280 by retracting the delivery catheter 204 and/or advancing the implantable device 280 beyond the distal terminus of the delivery catheter 204. This allows the implantable device 280 to partially expand toward an expanded or deployed state, while still being releasably secured to a distal portion of the implant shaft 206. In FIG. 2, for example, the implantable device 280 is shown in a partially-deployed position (also referred to as a “partially-deployed state” or “partially-deployed configuration”) in which the implantable device 280 has been advanced out of the distal portion of the delivery catheter 204, but is still secured to the implant shaft 206 to allow for controlled movement (e.g., linear, rotational) of and further deployment of the partially-deployed implantable device 280.

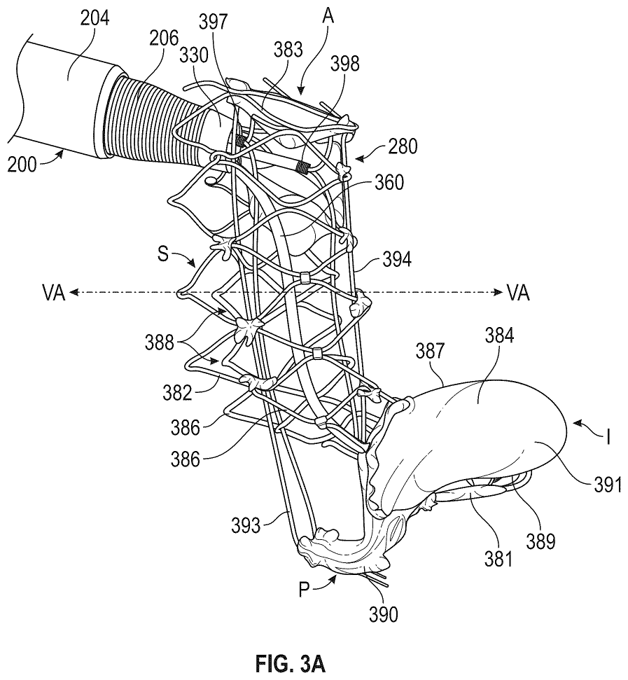

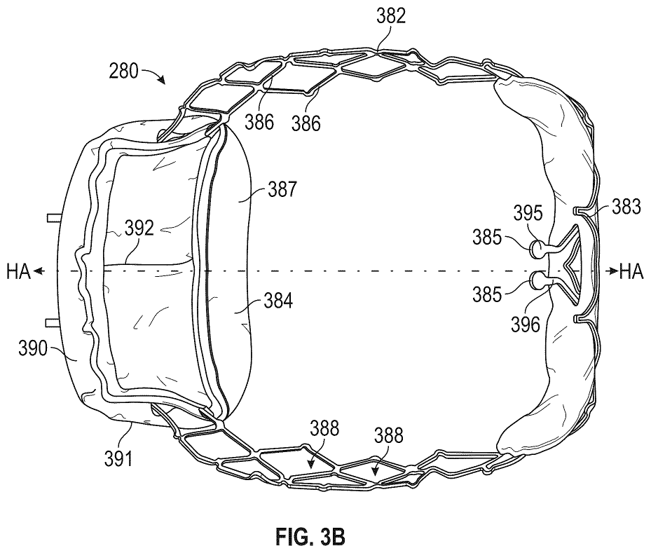

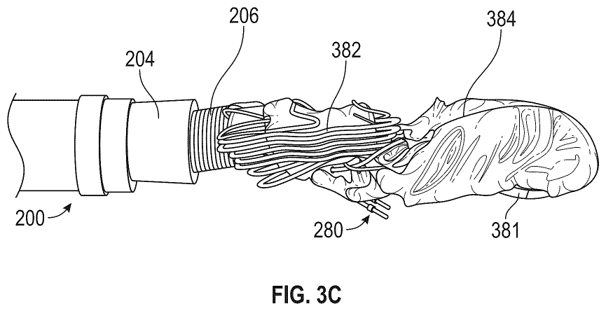

More specifically, FIG. 3A is an enlarged isometric side view of a distal portion of the delivery system 200 and the implantable device 280 in the partially-deployed position in accordance with embodiments of the present technology. In the illustrated embodiment, the implantable device 280 is a valve repair device having a fixation member 382 (also referred to as an “atrial fixation member,” an “anterior brim,” an “anterior-only brim,” an “anterior anchoring member,” a “suprannular anchoring member,” and/or the like) and a coaptation member 384 (also referred to as a “baffle”) extending from the atrial-fixation member 382 in a downstream direction. The atrial-fixation member 382 is configured to anchor the implantable device 280 to cardiac tissue proximate to a native mitral valve annulus and position the coaptation member 384 at a desired location with respect to the native valve anatomy of the heart. The implantable device 280 is designed to eliminate regurgitation by restoring physiologic coaptation in a diseased mitral valve. The coaptation member 384 can be covered in a biocompatible material (e.g., polytetrafluoroethylene (PTFE), expanded polytetrafluoroethylene (ePTFE)) and shaped (e.g., contoured) to fill a regurgitant orifice of the mitral valve from the posterior side and provide a new coaptation surface for the native leaflets of the mitral valve. The atrial-fixation member 382 can be flexible and can provide additional fixation and supra-annular stabilization. In the illustrated embodiment, the implantable device 280 further includes a posterior clip assembly 381 that can orient the implantable device 280 and provide sub-annular fixation.

In some embodiments, the implantable device 280 can include some features that are at least generally similar in structure and function, or identical in structure and function, to the corresponding features of one or more of the implantable devices described in (i) U.S. patent application Ser. No. 17/981,221, titled “CARDIAC VALVE REPAIR DEVICES, AND ASSOCIATED METHODS AND SYSTEMS,” and filed Nov. 4, 2022, (ii) U.S. patent application Ser. No. 16/044,447, titled “PROSTHETIC LEAFLET DEVICE,” and filed Jul. 24, 2018, (iii) International Patent Application No. PCT/US2018/061126, titled “LEAFLET EXTENSION FOR CARDIAC VALVE LEAFLET,” and filed Nov. 14, 2018, (iv) U.S. patent application Ser. No. 16/745,246, titled “IMPLANTABLE COAPTATION ASSIST DEVICES WITH SENSORS AND ASSOCIATED SYSTEMS AND METHODS,” and filed Jan. 16, 2020, (v) U.S. patent application Ser. No. 16/817,464, titled “CARDIAC VALVE REPAIR DEVICES WITH ANNULOPLASTY FEATURES AND ASSOCIATED SYSTEMS AND METHODS,” and filed Mar. 12, 2020, (vi) U.S. patent application Ser. No. 17/027,681, titled “VALVE REPAIR DEVICES WITH COAPTATION STRUCTURES AND MULTIPLE LEAFLET CAPTURE CLIPS,” and filed Sep. 21, 2020, and/or (vii) U.S. patent application Ser. No. 17/194,113, titled “DELIVERY SYSTEMS FOR CARDIAC VALVE DEVICES, AND ASSOCIATED METHODS OF OPERATION,” and filed Mar. 5, 2021, each of which is incorporated herein by reference in its entirety. Any of several prosthetic valve repair or replacement devices could similarly be used with delivery systems in accordance with the present technology, including complete mitral valve replacement devices. And, in addition to mitral valve devices, other valve repair or replacement devices could be delivered to the tricuspid, aortic, and pulmonic valves using delivery systems in accordance with the present technology.

FIG. 3B is a top view (e.g., an en face view) of the implantable device 280 in a deployed position and released (e.g., disconnected) from the delivery system 200 in accordance with embodiments of the present technology Referring to FIGS. 3A and 3B, the implantable device 280 is configured relative to a flow axis VA (FIG. 3A) in the direction of blood flow from the atrium to the ventricle and a transverse axis HA (FIG. 3B) at an angle (e.g., orthogonal) to the flow axis VA. As shown in FIG. 3A, the implantable device 280 has a posterior side portion P (e.g., a first side portion), an anterior side portion A (e.g., a second side portion), a superior end portion S (e.g., a first end portion), and an inferior end portion I (e.g., a second end portion).

The atrial-fixation member 382 can be formed of a mesh, such as a braid or laser-cut stent-like structure, including a plurality of interconnected wires or struts 386 which together define a plurality of openings or cells 388 (e.g., diamond-shaped openings) arranged in one or more rows (e.g., two rows). The struts 386 can be configured to self-expand from a collapsed delivery state when the implantable device 280 is positioned within the delivery catheter 204 (FIG. 3A) to the partially-deployed position shown in FIG. 3A and to the deployed position shown in FIG. 3B when released from the delivery system 200 (FIG. 3B). In the illustrated embodiment, the atrial-fixation member 382 comprises an anterior brim portion 383 at and/or proximate the anterior side portion A of the implantable device 280. The brim portion 383 can have a generally curved shape in a circumferential direction about the flow axis VA selected to engage the tissue above a native valve (e.g., a native mitral valve) when the implantable device 280 is implanted at the native valve. Moreover, in the illustrated embodiment the brim portion 383 extends only partially about the flow axis VA such that when the implantable device 280 is implanted at the native valve, the brim portion 383 only engages a portion (e.g., an anterior portion) of the circumference of the native tissue above the valve (e.g., the tissue of the left atrium).

Referring to FIG. 3B, the atrial-fixation member 382 can include one or more connectors 385 that are configured (e.g., sized, shaped, and/or positioned) to engage with one or more mating features on the delivery system 200, as described in greater detail below. The connectors 385 can extend from the struts 386 such that the connectors 385 are positioned between the superior end portion S (FIG. 3A) and the inferior portion S (FIG. 3A) of the implantable device 280. In the illustrated embodiment, the implantable device 280 includes two of the connectors 385, and the connectors 385 each include a key portion 395 (e.g., a circular portion) and an elongate portion 396 (e.g., an extension portion) connecting the key portion 395 to the struts 386 of the atrial-fixation member 382. In some aspects of the present technology, the connectors 385 can help maintain the implantable device 280 in a rotatably stable position as the clip assembly 381 is opened and closed.

Referring to FIGS. 3A and 3B, in the illustrated embodiment the coaptation member 384 extends away from the atrial-fixation member 382 along the flow axis VA, and at least a portion of the coaptation member 384 extends radially inward toward the flow axis VA to approximate a closed position of a native leaflet. The coaptation member 384 can be substantially stationary (e.g., little to no movement) during cardiac cycles such that the position of the coaptation member 384 relative to the atrial-fixation member 382 is at least substantially fixed in the deployed state. Thus, unlike native leaflets that move back and forth to open and close the native valve, the coaptation member 384 remains stationary during diastole and systole.

The coaptation member 384 can have an anterior portion 387 with a smooth, atraumatic surface for coapting with at least a portion of one or more native leaflets and a posterior portion 389 (FIG. 3A) configured to displace and, optionally, engage at least a portion of another native leaflet. In the illustrated embodiment, the coaptation member 384 includes a brim member 390 (also referred to as a “posterior brim,” a “posterior extension,” and/or the like) that may be separate from and coupled to, or integral with the coaptation member 384. The brim member 390 extends away from a portion (e.g., a superior-posterior portion) of the coaptation member 384 in a superior-posterior direction. The brim member 390 can be flexible. For example, the brim member 390 can comprise a nitinol wire form covered with PET fabric to provide a platform for long term tissue incorporation into the brim member 390. When the implantable device 280 is implanted at a native valve, the brim member 390 can extend above the native valve annulus into the atrium (e.g., the left atrium) above the valve and can contact a portion (e.g., a posterior portion) of the native tissue above the valve (e.g., the tissue of the left atrium). In some aspects of the present technology, the brim member 390 is relatively small to reduce (e.g., minimize) undesirable interaction of the implantable device 280 with the left atrium that may otherwise move or shift the implantable device relative to the native valve.

The coaptation member 384 can be made from a plurality of struts that form a basket-like or frame-like structure (e.g., a mesh structure, a laser cut stent frame) with an at least partially hollow interior and a covering 391 extending over at least a portion of the struts to provide a smooth suitable surface for coaptation at the anterior portion 387. The covering 391 can comprise a slit 392 (FIG. 3B) at a superior (e.g., top) portion of the coaptation member 384 to allow for one or more components of the delivery system 200 to attach to the coaptation member 384 and/or the clip assembly 381. The slit 392 can be configured to close after the coaptation member 384 is detached from the delivery system 200.

Referring to FIG. 3A, the clip assembly 381 depends from the posterior portion 389 of the coaptation member 384 and can be opened to extend behind the native leaflet that the coaptation member 384 displaces. The clip assembly 381 may grasp the native leaflet and/or engage sub-annular cardiac tissue for sub-annular stabilization of the implantable device 280. In some embodiments, for example, the clip assembly 381 reaches under the central portion (i.e., P2) of the posterior leaflet up to the sub-annular space.

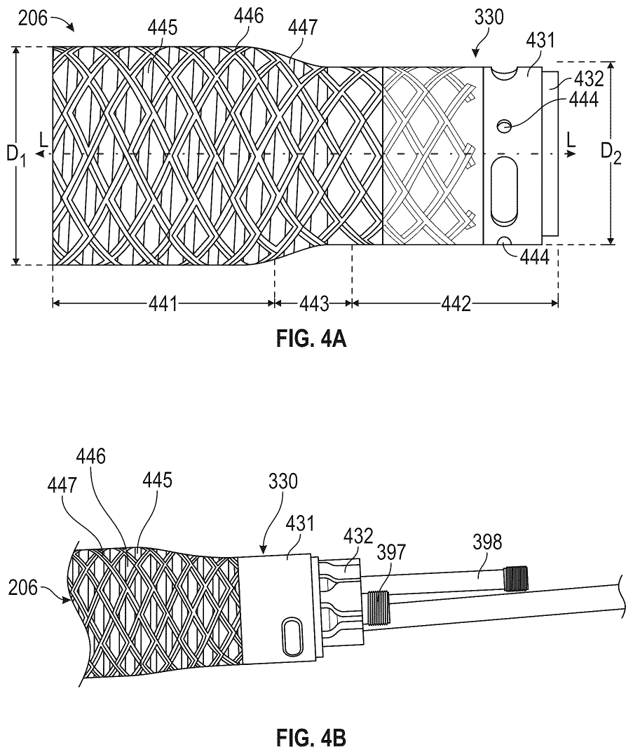

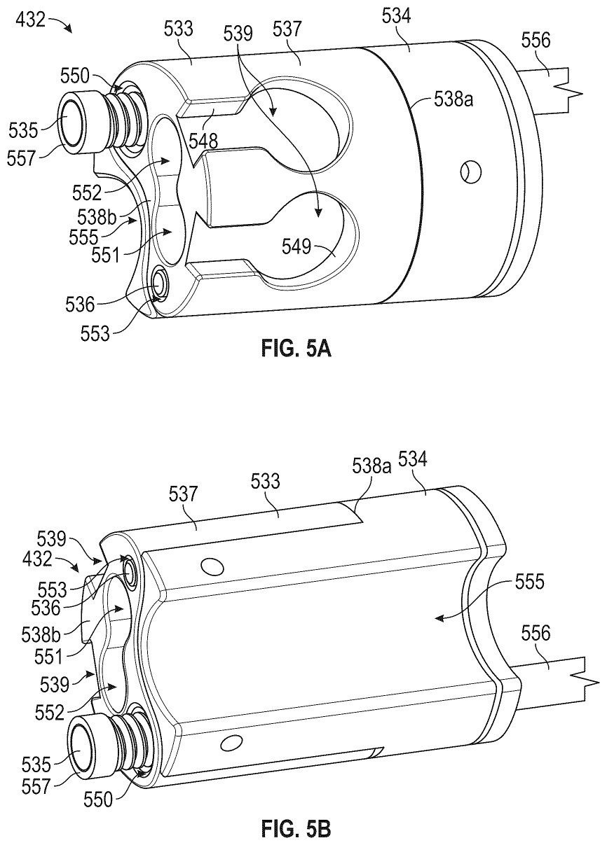

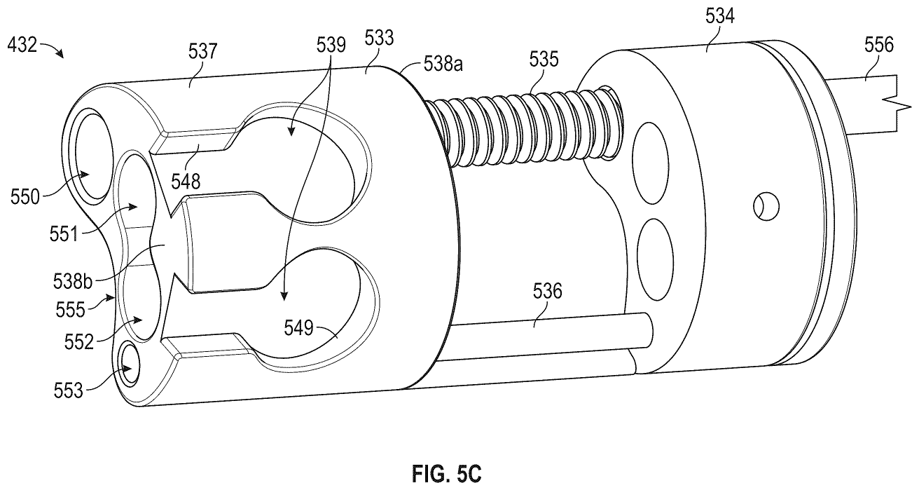

In the illustrated embodiment, the implant shaft 206 includes a distal hub assembly 330 that releasably engages the connectors 385 (FIG. 3B) of the implantable device 280, as described in detail below with reference to FIGS. 4A-5C. Accordingly, the implant shaft 206 can be moved/steered (e.g., rotated, translated) to correspondingly move the implantable device 280 when it is deployed from the delivery catheter 204. The hub assembly 330 includes a first (e.g., upper) cinching post 397 and a second (e.g., lower) cinching post 398. The first cinching post 397 is configured to receive a first cinching line 393 (e.g., suture) that is routed at least partially around a superior portion of the atrial-fixation member 382 and/or the brim member 390 through corresponding eyelets and/or suture loops coupled thereto or integrally formed therewith. Likewise, the second cinching post 398 is configured to receive a second cinching line 394 that is routed at least partially around an inferior portion of the atrial-fixation member 382 and/or the brim member 390 through corresponding eyelets and/or suture loops coupled thereto or integrally formed therewith. The first and second cinching lines 393, 394 can extend from the implantable device 280 through the implant shaft 206 to the implant handle 216 (FIG. 2).

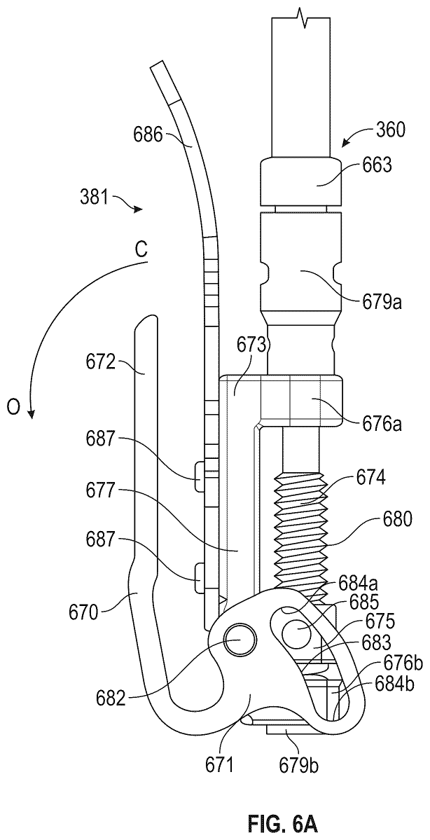

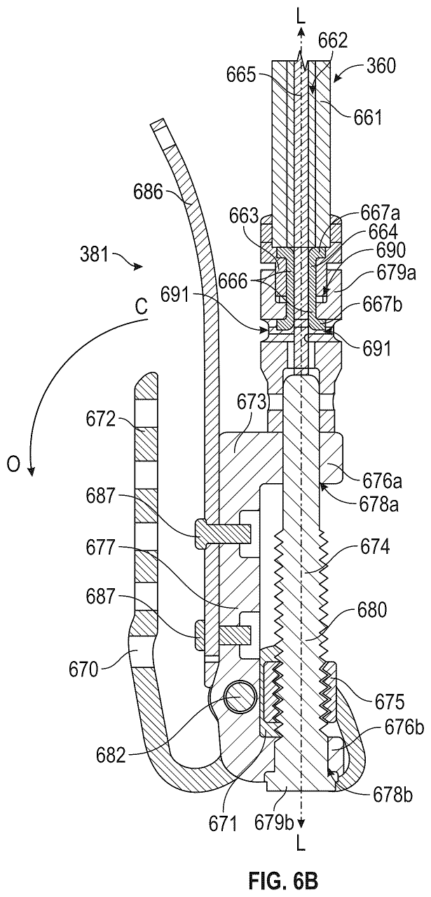

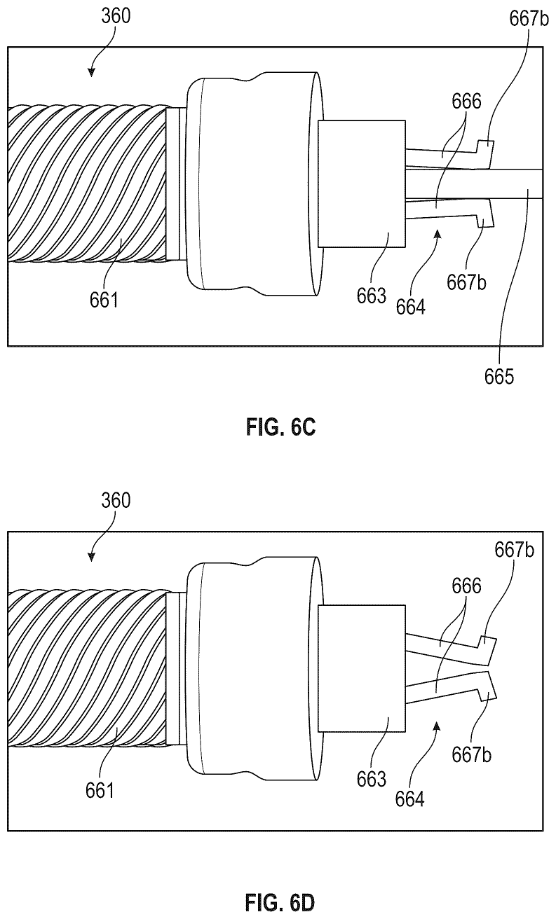

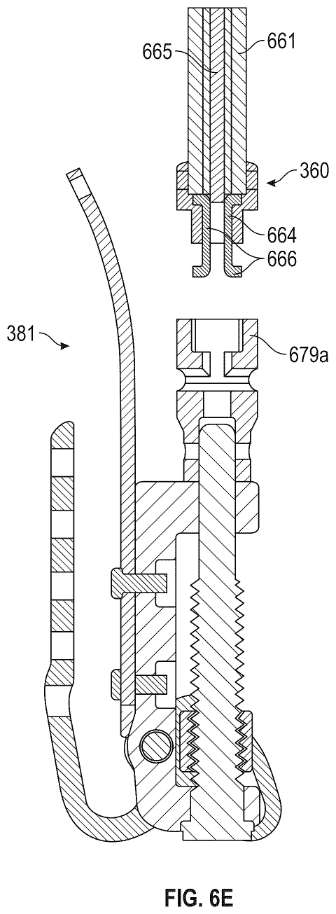

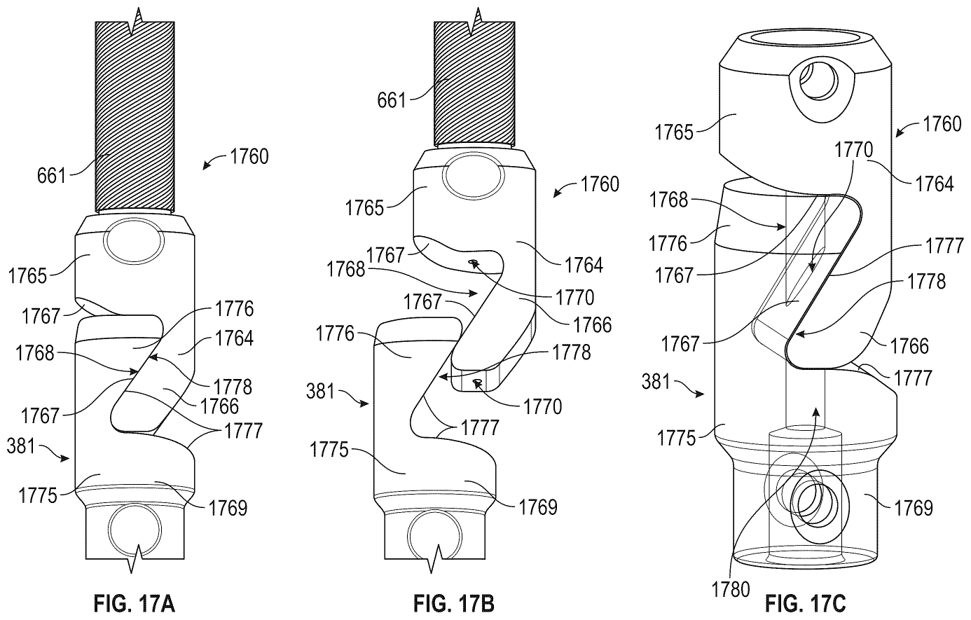

In the illustrated embodiment, the delivery system 200 further includes a clip cable assembly 360 extending from the implant handle 216 (FIG. 2) through the implant shaft 206 and that releasably engages the clip assembly 381, as described in further detail below with reference to FIGS. 6A-6E and FIGS. 17A-17C. More specifically, the clip cable assembly 360 can extend distally from the hub assembly 330 and through the slit 392 (FIG. 3B) into the coaptation member 384 for engaging a drive head of the clip assembly 381. The clip cable assembly 360 can be actuated (e.g., rotated) via the implant handle 216 (FIG. 2) to drive the clip assembly 381 to open and/or close, as described in further detail below with reference to FIGS. 6A-6E, 9, 11A, and 11B. The clip cable assembly 360 can be flexible.

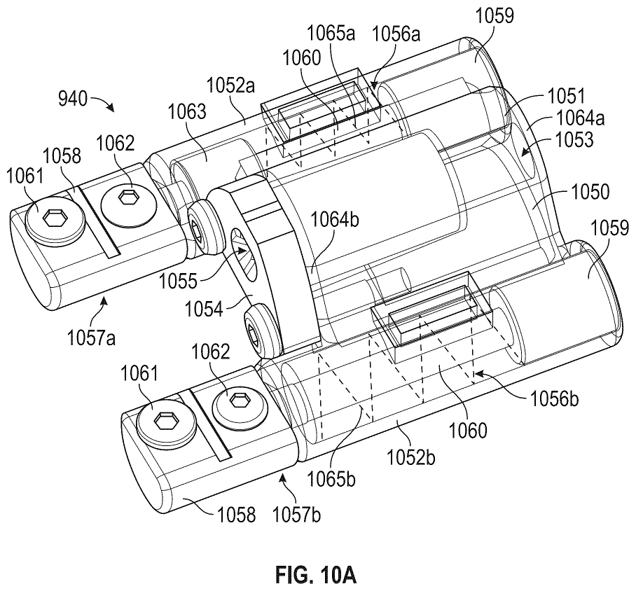

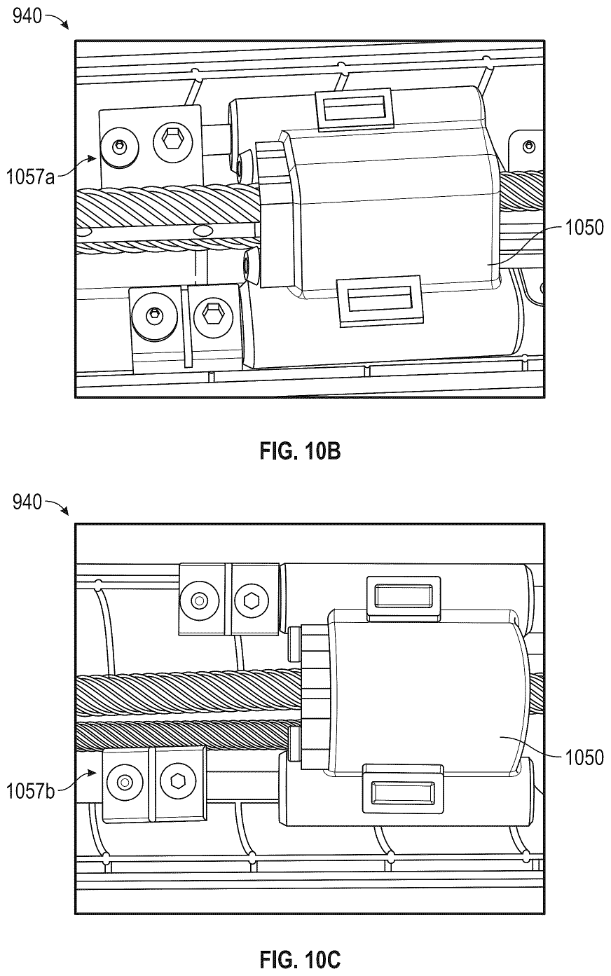

The first and second cinching lines 393, 394 can be tensioned (e.g., pulled proximally) via the implant handle 216 (FIG. 2) to at least partially cinch and collapse the implantable device 280, as described in further detail below with reference to FIGS. 10A-10C and 12A-13C. Likewise, the first and second cinching lines 393, 394 can be relaxed (e.g., moved distally) via the implant handle 216 (FIG. 2) to permit the implantable device 280 to expand, as also described in further detail below with reference to FIGS. 10A-10C and 12A-13C. More specifically, FIG. 3A shows the implantable device 280 in an uncinched position. FIG. 3C is an enlarged isometric side view of the distal portion of the delivery system 200 and the implantable device 280 in the partially-deployed position with the atrial-fixation member 382 in a cinched position in accordance with embodiments of the present technology. Referring to FIG. 3A, in the uncinched position, the coaptation member 384 is expanded laterally away from a longitudinal axis of the implant shaft 206 such that the clip cable assembly 360 extends laterally from the hub assembly 330 to the clip assembly 381. In contrast, referring to FIG. 3B, in the cinched position, the coaptation member 384 is generally aligned with the longitudinal axis of the implant shaft 206 such that the clip cable assembly 360 (obscured by the implantable device in FIG. 3C) extends along a generally straight path from the hub assembly 330 to the clip assembly 381.

FIGS. 4A and 4B are enlarged side views of a distal portion of the implant shaft 206 including the hub assembly 330 in accordance with embodiments of the present technology. Referring to FIG. 4A, the implant shaft 206 extends along a longitudinal axis L and includes a proximal portion 441 having a first diameter D₁, a distal portion 442 having a second diameter D₂ less than the first diameter D₁, and a transition region 443 between the proximal portion 441 and the distal portion 442 in which the diameter of the implant shaft 206 tapers from the first diameter to the second diameter D₂. The hub assembly 330 can be secured to the implant shaft 206 at the distal portion 442 via, for example, welds, bonds, or fasteners 444. The first diameter D₁ can be selected to match or substantially match an inner diameter of the delivery catheter 204 (FIG. 3A) to provide a snug fit therebetween to, for example, ensure that the delivery catheter 204 and the implant shaft 206 having matching flex radii. The smaller second diameter D₂ can provide space for a portion of atrial-fixation member 382 to be packed over the distal portion 442 within the delivery catheter 204, as described in greater detail below.

The implant shaft 206 can comprise a wire 445 coiled about the longitudinal axis L (FIG. 4A) of the implant shaft 206, a mesh or braid 446 of filaments extending over the wire 445, and one or more liner and/or jacket layers 447 extending over and/or under the wire 445 and the braid 446. The wire 445 and the braid 446 can comprise stainless steel and/or other suitable materials. The layers 447 can comprise a plastic material, elastomeric material, and/or thermoplastic elastomer (TPE) material that encapsulates (e.g., fully encapsulates) the wire 445 and the braid 446. In some embodiments, the layers 447 can be formed from a TPE manufactured by Arkema S.A., of Colombes, France, such as the TPEs manufactured under the trademark “Pebax.” In some embodiments, the wire 445 and/or the braid 446 are secured (e.g., welded, fastened, bonded) to the hub assembly 330 at the distal portion 442.

In the illustrated embodiment, the hub assembly 330 includes an outer hub component 431 and an inner hub component 432. The outer hub component 431 can be fixed to the implant shaft 206, and the inner hub component 432 can be movably coupled to the outer hub component 431. The connectors 385 (FIG. 3B) can be secured between the outer hub component 431 and the inner hub component 432 in a first position, and the inner hub component 432 can be moved (e.g., translated) distally relative to outer hub component 431 to release the connectors 385 from between the outer hub component 431 and the inner hub component 432 to release the implantable device (FIGS. 3A and 3B) from the hub assembly 330.

FIGS. 5A and 5B are isometric side views of the inner hub component 432 in a retracted position (e.g., a coupled position) in accordance with embodiments of the present technology. FIG. 5C is an isometric side view of the inner hub component 432 in an extended position (e.g., a release position) in accordance with embodiments of the present technology. Referring to FIGS. 5A-5C, the inner hub component 432 includes a first housing member 533 movably (e.g., slidably) coupled to a second housing member 534 via a leadscrew 535 and a post 536. In some embodiments, the second housing member 534 can be fixed (e.g., secured) to the outer hub component 431 (FIGS. 4A and 4B). The first housing member 533 includes an outer surface 537, a proximal side or edge 538a, and a distal side or edge 538b. In the illustrated embodiment, the outer surface 537 of the first housing member 533 includes multiple recesses 539 that extend from the distal edge 538b toward the proximal edge 538a. The recesses 539 are configured (e.g., shaped, sized, and/or positioned) to receive and secure corresponding ones of the connectors 385 of the implantable device 280 (FIG. 3B). Referring to FIGS. 3B, 5A, and 5C, in the illustrated embodiment the recesses 539 each include (i) a first portion 548 having a generally elongate (e.g., rectangular) shape for receiving the elongate portion 396 of a corresponding one of the connectors 385 and (ii) a second portion 549 having a generally circular shape for receiving the key portion 395 of a corresponding one of the connectors 385. In other embodiments, the recesses 539 can have other suitable shapes for receiving and securing the connectors 385.

Referring to FIGS. 5A-5C, the first housing member 533 and the second housing member 534 (collectively “housing members 533, 534”) define a first lumen 550 configured to receive the leadscrew 535, a second lumen 551 configured to receive the first cinching post 397 (FIGS. 3A and 4B) and the first cinching line 393 (FIG. 3A) therein/therethrough, and a third lumen 552 configured to receive the second cinching post 398 (FIGS. 3A and 4B) and the second cinching line 394 (FIG. 3A) therein/therethrough. In some embodiments, the first lumen 550 includes a threaded inner surface configured to engage (e.g., mate) with a threaded outer surface of the leadscrew 535. In some embodiments, the second and third lumens 551, 552 can be connected such that the second and third lumens 551, 552 collectively have a lemniscate cross-sectional shape. The first housing member 533 can further define a fourth lumen 553 configured to slidably receive the post 536. The post 536 can help constrain the first housing member 533 to only translate distally/proximally relative to the second housing member 534. In some embodiments, the post 536 is fixedly secured to the second housing member 534. Moreover, the second housing member 534 can define a channel 555 configured to receive the clip cable assembly 360 (FIG. 3A). Accordingly, in the retracted position shown in FIGS. 5A and 5B, the housing members 533, 534 can collectively have a kidney-like cross-sectional shape (e.g., a non-circular cross-sectional shape, a crescent-like cross-sectional shape).

In the illustrated embodiment, the leadscrew 535 is operably coupled to a drive shaft 556. The drive shaft 556 can be coupled to the leadscrew 535 via welding, adhesives, fasteners, and/or other suitable types of connections, or the drive shaft 556 and the leadscrew 535 can be manufactured from a single piece of wire or rod. The drive shaft 556 can extend from the leadscrew 535 proximally through the implant shaft 206 (FIGS. 2 and 3A-4B) to the implant handle 216 (FIG. 2). In the illustrated embodiment, the leadscrew 535 includes a stop member 557 configured (e.g., sized and/or shaped) to engage a stop surface (e.g., a stepped surface; obscured in FIGS. 5A-5C) within the first lumen 550 when the inner hub component 432 is in the extended position to inhibit further extension of the first housing member 533 relative to the second housing member 534.

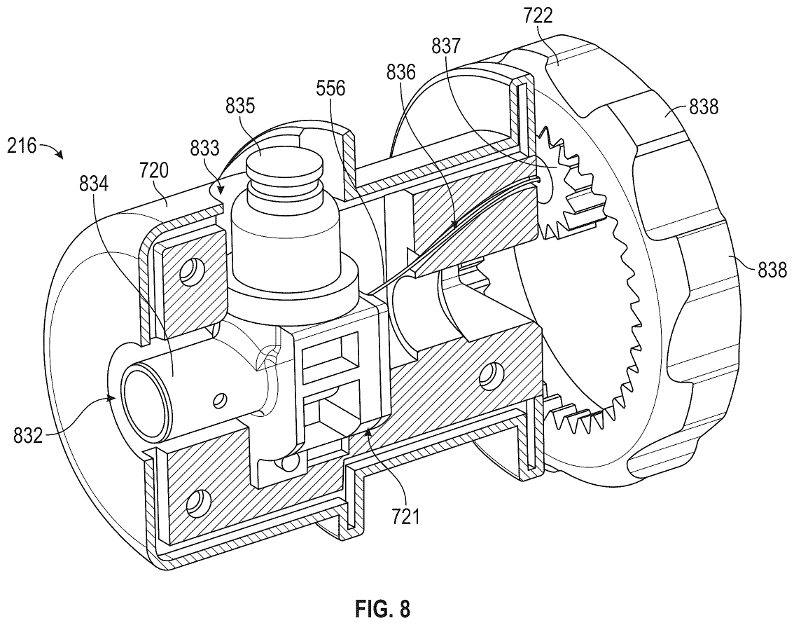

Referring to FIGS. 3A-5C together, the atrial-fixation member 382 of the implantable device 280 can be secured to the implant shaft 206 via the hub assembly 330 by positioning the connectors 385 within corresponding ones of the recesses 539 of the first housing member 533 of the inner hub component 432 with the inner hub component 432 in the retracted position (FIGS. 5A and 5B). More specifically, the outer hub component 431 can secure/restrain the connectors 385 within the recesses 539 in the retracted position. To release the atrial-fixation member 382 from the hub assembly 330 and the implant shaft 206, the drive shaft 556 can be actuated (e.g., rotated) via the implant handle 216 (FIG. 2), as described in greater detail below with reference to FIG. 8, to rotate the leadscrew 535 to drive the first housing member 533 of the inner hub component 432 distally relative to the second housing member 532 and the outer hub component 431 to the extended position (FIG. 5C). As the recesses 539 move distally past/out of the outer hub component 431, the outer hub component 431 no longer constrains the connectors 385 such that they can flex outward from the recesses 539—thereby releasing the atrial-fixation member 382 from the hub assembly 330 and the implant shaft 206.

Referring to FIGS. 3A and 3B, the connectors 385 extend from a portion of the atrial-fixation member 382 positioned between the superior end portion S and the inferior end portion I of the implantable device 280 (e.g., via a mid-brim attachment region or mid-fixation member attachment region). Accordingly, referring to FIGS. 3A-4B, when the connectors 385 are secured to the hub assembly 330, at least a portion of the superior end portion S of the atrial-fixation member 382 can overlap the distal portion 442 of the implant shaft 206 and the hub assembly 330. The smaller second diameter D₂ of the distal portion 442 provides space that allows the implantable device 280 to be compressed within the delivery catheter 204 with such an overlap. In some aspects of the present technology, this arrangement increases the overall flexibility of the distal portion of the delivery system 200. For example, the hub assembly 330 and the implantable device 280 can be relatively stiffer than the implant shaft 206 when positioned within the delivery catheter 204. Accordingly, overlapping the superior end portion S of the atrial-fixation member 382 over the hub assembly 330 can reduce the overall stiffness provided by these components compared to, for example, positioning the hub assembly 330 entirely or substantially proximal to the atrial-fixation member 382. That is, overlapping the relatively rigid atrial-fixation member 382 over the hub assembly 330 within the delivery catheter 204 can reduce an overall rigid length of material within the delivery catheter 204 proximal to the atrial-fixation member 382. In some embodiments, the distal portion 442 of the implant shaft 206 can be about 10 millimeters, thereby reducing the overall rigid length by about 10 millimeters. The improved flexibility and reduced stiff length can provide improved maneuverability of the delivery catheter 204 within tight anatomies, such as within patients having a narrow and/or short atrium, requiring a low septal puncture height, etc.

In additional aspects of the present technology, securing the hub assembly 330 to the connectors 385 at a middle region of the atrial-fixation member 382 can improve controllability of the inferior end portion I of the atrial-fixation member 382 via the implant shaft 206. For example, the mid-atrial-fixation member attachment region can reduce the distance between the implant shaft 206 and the inferior end portion I of the atrial-fixation member 382 to provide a more rigid connection that provides for better control of the implantable device 280.

FIGS. 6A and 6B are a side view and a side cross sectional view, respectively, of the clip assembly 381 of FIGS. 3A and 3B coupled to a distal portion of the clip cable assembly 360 in a locked position (e.g., a coupled position) in accordance with embodiments of the present technology. In some embodiments, the clip assembly 381 can include some features that are at least generally similar in structure and function, or identical in structure and function, to corresponding features of one or more of the clip assemblies described in U.S. patent application Ser. No. 17/981,221, titled “CARDIAC VALVE REPAIR DEVICES, AND ASSOCIATED METHODS AND SYSTEMS,” and filed Nov. 4, 2022, which is incorporated herein by reference in its entirety.

Referring to FIGS. 6A and 6B, in the illustrated embodiment the clip assembly 381 comprises (i) a clip member 670 having a root portion 671 and an arm portion 672 extending from the root portion 671, (ii) a back member 673 configured to be positioned within the coaptation member 384 (FIGS. 3A and 3B) of the implantable device 280, (iii) a threaded member 674, and (iv) an actuation member 675. The back member 673 has a first (e.g., upper) horizontal portion 676a and a second (e.g., lower) horizontal portion 676b connected to respective end portions of an elongate vertical portion 677 and having first and second apertures 678a-b (FIG. 6B), respectively, extending vertically therethrough. In the illustrated embodiment, the threaded member 674 has (i) a first (e.g., upper) head 679a positioned above the first horizontal portion 676a, (ii) a second (e.g., lower) head 679b rotatably retained in the second aperture 678b (FIG. 6B) in the second horizontal portion 676b, and (iii) a partially threaded rod 680 extending between the first and second heads 679a-b and at least partially through the first and second apertures 678a-b (FIG. 6B) in the first and second horizontal portions 676a-b, respectively. The root portion 671 of the clip member 670 is pivotably coupled to the vertical portion 677 of the back member 673 via, for example, a pin 682.

Referring to FIG. 6A, the root portion 671 further includes a slot 683 having a first end portion 684a and a second end portion 684b. In some embodiments, the slot 683 can have a V-shape that is, in the closed configuration shown in FIG. 6A, angled off vertical (e.g., angled downward relative to the threaded member 674 and/or the vertical portion 677 of the back member 673). The actuation member 675 can be a threaded nut secured to the threaded portion of the rod 680 and having a projection 685 extending into the slot 683 of the root portion 671.

Referring to FIGS. 6A and 6B, in the illustrated embodiment the clip assembly 381 further includes an attachment member 686 (also referred to as a “spider member”) coupled to the vertical portion 677 of the back member 673 opposite the threaded member 674. For example, the attachment member 686 can be secured via rivets 687 to the back member 673. The attachment member 686 can include multiple arm or finger portions that splay outward relative to the back member 673 (e.g., into and/or out of the page in FIGS. 6A and 6B). The attachment member 686 is configured to be secured to the coaptation member 384 (FIGS. 3A and 3B) of the implantable device 280 such that the clip assembly 381 is fixedly attached to the coaptation member 384. In some embodiments, the attachment member 686 can be secured to the coaptation member 384 via suturing.

In the illustrated embodiment, the clip cable assembly 360 comprises (i) a clip cable 661 defining a lumen 662 (FIG. 6B), (ii) a torque member 663 fixedly coupled to the clip cable 661, (iii) a lock member 664 (FIG. 6B) coupled to the torque member 663, and (iv) a lockwire 665 (FIG. 6B) slidably positioned within the lumen 662 and configured to extend through the torque member 663 and the lock member 664 when the clip cable assembly 360 is coupled to the clip assembly 381 as shown in FIGS. 6A and 6B. The lockwire 665 can extend proximally through the clip cable 661 to the implant handle 216 (FIG. 2).

FIGS. 6C and 6D are perspective side views of the clip cable assembly 360 in a locked position and an unlocked position, respectively, in accordance with embodiments of the present technology. Referring to FIGS. 6B-6D, the lock member 664 can comprise one or more (e.g., two) flexible arms 666 each having a proximal end portion 667a (FIG. 6B) secured to (e.g., fixed to) the clip cable 661 and/or the torque member 663 and a distal end portion 667b comprising a projection that extends, for example, at least partially laterally relative to a longitudinal axis L (FIG. 6B) of the lockwire 665 and the threaded member 674 of the clip assembly 381. The arms 666 can be formed from nitinol and/or another suitable material and can be biased to flex inward toward the longitudinal axis L when the lockwire 665 is not positioned therebetween in the unlocked position as shown in FIG. 6D. The clip cable 661 can comprise a multi-layer (e.g., three-layer) torque cable formed of stainless steel and/or another suitable material. The torque member 663 and the lockwire 665 can comprise nitinol, stainless steel, and/or other suitable materials. In some embodiments, the torque member 663 is welded to the clip cable 661.

Referring to FIG. 6B, the first head 679a can include/define a drive recess 690 for releasably receiving the torque member 663. The drive recess 690 can be a slotted recess, a hex recess, a square recess, a rectangular recess, and/or the like that matches a shape of the torque member 663 such that torque (e.g., rotation) applied to the torque member 663 is transmitted to the first head 679a. For example, in the embodiment shown in FIGS. 6C and 6D, the torque member 663 has a rectangular cross-sectional shape and the drive recess 690 can have a corresponding rectangular cross-sectional shape. The first head 679a can further include/define one or more recesses or apertures 691 extending laterally away from the longitudinal axis L and configured (e.g., shaped, size) to receive the second portions 687b of corresponding ones of the arms 666 in the locked position shown in FIG. 6B.

Accordingly, to lock the clip cable assembly 360 to the clip assembly 381, the torque member 663 can be inserted (e.g., press fit or slip fit) into the drive recess 690 and the arms 666 positioned proximate to the apertures 691 with the lockwire 665 removed from between the arms 666 (e.g., as shown in FIG. 6D). The lockwire 665 can then be moved distally through the lumen 662 of the clip cable 661 and between the arms 666 of the lock member 664 to flex the arms 666 radially outward such that the distal end portions 667b are secured within corresponding ones of the apertures 691. To subsequently unlock the clip cable assembly 360 from the clip assembly 381, the lockwire 365 can be withdrawn proximally from between the arms 666 to permit the arms 666 to flex radially inward toward the longitudinal axis L out of the apertures 691, and the clip cable assembly 360 can be withdrawn proximally relative to the clip assembly 381 to remove the torque member 663 from the drive recess 690. For example, FIG. 6E is a cross-sectional side view of the clip assembly 381 decoupled from the clip cable assembly 360 in an unlocked position in accordance with embodiments of the present technology. In the illustrated embodiment, the lockwire 665 is withdrawn proximally from between the arms 666, and the clip cable assembly 360 is withdrawn proximally from the first head 679a of the clip assembly 381.

Referring to FIGS. 6A and 6B, when the clip cable assembly 360 is coupled to the clip assembly 381, the clip cable assembly 360 can be driven (e.g., rotated) to move the clip assembly 381 between a closed position shown in FIGS. 6A and 6B and an open position. For example, in the closed position, the projection 685 is positioned at or proximate the first end portion 684a of the slot 683. To actuate the clip assembly 381 to open the clip member 670 to the open position—e.g., to pivot the arm portion 672 away from the attachment member 686 in the direction of arrow O—the clip cable assembly 360 can be rotated in a first direction to rotate the threaded member 674 in the first direction via the engagement of the torque member 663 with the drive recess 690 in the first head 679a. Rotation of the threaded member 674 in the first direction drives the actuation member 675 upward toward the first horizontal portion 676a along the threads of the rod 680. As the actuation member 675 moves upward, the projection 685 moves along the slot 683 from the first end portion 684a toward the second end portion 684b and drives the root portion 671 to pivot about the pin 682 in a clockwise direction and thus the arm portion 672 to pivot open in the direction of the arrow O. The clip assembly 381 can reach a fully open position when the projection 685 reaches the second end portion 684b of the slot 683. In some embodiments, the arm portion 672 can pivot open by about 90° relative to the back member 673 and the attachment member 686. The second end portion 684b can function as a hard stop that prevents further opening of the clip member 670.

To actuate the clip assembly 381 to close the clip member 670—e.g., to pivot the arm portion 672 toward the attachment member 686 in the direction of arrow C—the clip cable assembly 360 can be rotated in a second direction opposite the first direction to rotate the threaded member 674 in the second direction via the engagement of the torque member 663 with the drive recess 690 in the first head 679a. Rotation of the threaded member 674 in the second direction drives the actuation member 675 downward toward the second horizontal portion 676b along the threads of the rod 680. As the actuation member 675 moves downward, the projection 685 moves along the slot 683 from the second end portion 684b toward the first end portion 684a and drives the root portion 671 to pivot about the pin 682 in a counterclockwise direction and thus the arm portion 672 to pivot closed in the direction of the arrow C.

FIGS. 17A and 17B are perspective side views of a clip cable assembly 1760 in a locked position and an unlocked position, respectively, relative to the clip assembly 381 in accordance with additional embodiments of the present technology. FIG. 17C is a partially-transparent side view of the clip cable assembly 1760 and the clip assembly 381 of FIGS. 17A and 17B in the locked position in accordance with embodiments of the present technology. The clip cable assembly 1760 can function similarly to the clip cable assembly 360 described in detail above with reference to FIGS. 6A-6E and, accordingly, although reference is frequently made to the clip cable assembly 360, aspects of the present technology can alternatively be implemented with the clip cable assembly 1760.

Referring to FIGS. 17A-17C, in the illustrated embodiment, the clip cable assembly 1760 comprises (i) the clip cable 661 (FIGS. 17A and 17B) defining the lumen 662 (obscured in FIGS. 17A and 17C; shown in FIG. 6B), (ii) a lock and/or torque member 1764 (referred to as the “lock member 1764”) fixedly coupled to the clip cable 661 (e.g., to a distal portion of the clip cable 661), and (iii) the lockwire 665 (obscured in FIGS. 17A and 17C; shown in FIG. 6B) slidably positioned within the lumen 662 and configured to extend through the lock member 1764 when the clip cable assembly 1760 is coupled to the clip assembly 381 as shown in FIGS. 17A and 17C. In some embodiments, the lock member 1764 has an S-like shape including a base 1765 and a projection or arm 1766 extending from the base 1765. The base 1765 and the arm 1766 together have an engagement surface 1767 defining a recess 1768. Referring to FIGS. 17B and 17C, the lock member 1764 can include a lumen 1770 (i) extending through the base 1765 and through the arm 1766 and (ii) opening to the recess 1768. That is, the lumen 1770 can comprise a first lumen or through hole extending through the base 1765 and aligned with the lumen 662 (FIG. 6B) of the clip cable 661, and a second lumen or through hole extending through the arm 1766 and that is aligned with the first through hole.

Referring to FIGS. 17A-17C, the clip assembly 381 includes a head 1769 (e.g., a screw cap) fixedly coupled to the threaded member 674 (FIGS. 6A and 6B) that is actuatable (e.g., rotatable) to open and close the clip member 670 (FIGS. 6A and 6B) as described in detail above with reference to FIGS. 6A-6E. In the illustrated embodiment, the head 1769 has an S-like shape that matches/corresponds to the S-like shape of the lock member 1764. In particular, the head 1769 can have an S-like shape including a base 1775 and a projection or arm 1776 extending from the base 1775. The base 1775 and the arm 1776 together have an engagement surface 1777 defining a recess 1778. Referring to FIG. 17C, the head 1769 can include a lumen 1780 (i) extending through the base 1775 and through the arm 1776 and (ii) opening to the recess 1778. That is, the lumen 1780 can comprise a first lumen or through hole extending through the base 1775, and a second lumen or through hole extending through the arm 1776 and that is aligned with the first through hole. The lock member 1764 and the head 1769 can be formed of stainless steel and/or other suitable materials, and may be electropolished.

Referring to FIGS. 17A and 17C, to lock the clip cable assembly 1760 to the clip assembly 381, the lock member 1764 can be mated with the head 1769 and the lockwire 665 (FIG. 6B) can be inserted through the lumen 662 of the clip cable 661 (FIG. 6B), through the lumen 1770 of the lock member 1764, and through the lumen 1780 of the head 1769. More particularly, the S-shapes of the lock member 1764 and the head 1769 can be fit together such that the arm 1766 of the of lock member 1764 is positioned within the recess 1778 of the head 1769 and the arm 1776 of the head 1769 is positioned within the recess 1768 of the lock member 1764. In some embodiments, in the locked position shown in FIGS. 17A and 17C, the engagement surface 1767 of the lock member 1764 at least partially engages/contacts the engagement surface 1777 of the head 1769. In this position, the lumen 1770 of the lock member 1764 is aligned with the lumen 1780 of the head 1769 such that, when the lockwire 665 is inserted through, the lock member 1764 and the head 1769 are fixedly coupled together. When the clip cable assembly 1760 is coupled to the clip assembly 381, the clip cable assembly 1760 can be driven (e.g., rotated) to rotate the head 1769 to move the clip assembly 381 between a closed position shown in FIGS. 6A and 6B and an open position.

To subsequently unlock the clip cable assembly 1760 from the clip assembly 381, the lockwire 665 (FIG. 6B) can be withdrawn proximally from the lumen 1780 of the head 1769 and the lumen 1770 of the lock member 1764. As shown in FIG. 17B, once the lockwire 665 is withdrawn, the lock member 1764 is free to disengage the head 1769. More specifically, the arm 1766 of the lock member 1764 can move out of the recess 1778 of the head 1769 with, for example, the engagement surface 1767 of the lock member 1764 slidably disengaging the engagement surface 1777 of the head 1769.

Accordingly, in some embodiments the lock member 1764, the head 1769, and the lockwire 665 (FIG. 6B) together comprise a release mechanism that releasably secures the clip assembly 381 to the clip cable assembly 1760 to allow for actuation of the clip assembly 381 and subsequent release of the clip assembly 381. In some aspects of the present technology, the illustrated mechanism requires only two components and less maneuvering of the catheters prior to release, as smooth release can occur from a wide variety of angulations. Accordingly, the release mechanism can be user-friendly and easy to manufacture.

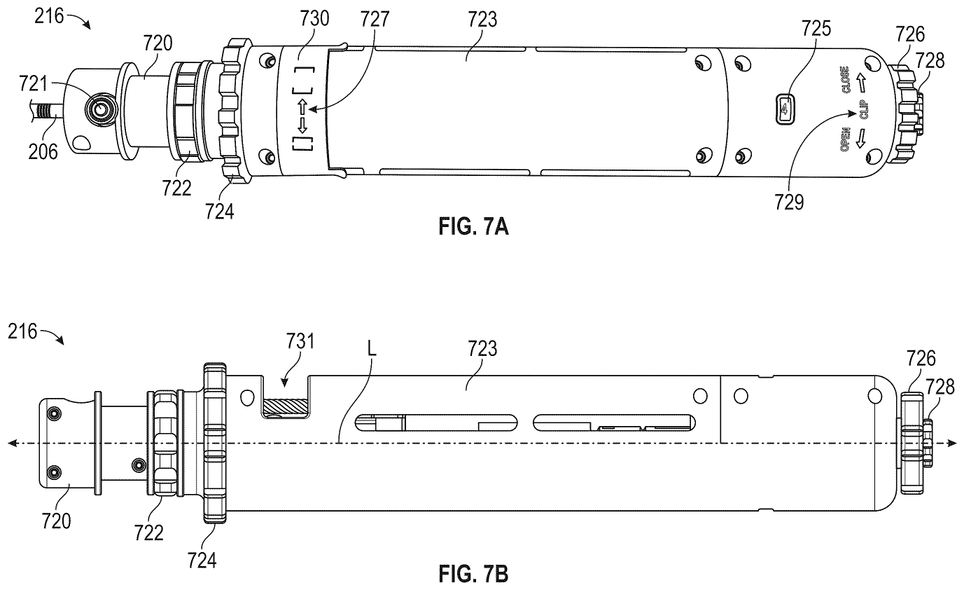

FIGS. 7A and 7B are a top view and a side view, respectively, of the implant handle 216 of FIG. 2 in accordance with embodiments of the present technology. Referring to FIGS. 7A and 7B, the implant handle 216 includes a connector 720 coupled (e.g., fixed) to the implant shaft 206 (FIG. 7A) and a housing 723 coupled to the connector 720. Accordingly, movement (e.g., proximal and/or distal translation and/or rotation) of the implant handle 216 can correspondingly move the implant shaft 206. As described in further detail below with reference to FIG. 8, the connector 720 can include a connector assembly 721 (FIG. 7A) configured to be coupled to the implant shaft 206. The connector assembly 721 can comprise one or more adaptors, valves, sealing members, and/or fluid control components for, for example, maintaining hemostasis, facilitating the ingress/egress of fluids (e.g., blood, priming solution, saline) into the implant shaft 206, etc. In some embodiments, the housing 723 can include a removable cover 730 (FIG. 7A) that can be secured over an opening 731 (FIG. 7B) in the housing 723 via a snap-fit or other arrangement. The cover 730 can be detached from the housing 723 to provide user access to the first and second cinching lines 393, 394 (FIG. 3A). More specifically, the cover 730 can provide user access to cut and/or remove the first and second cinching lines 393, 394. The components of the implant handle 216 can be formed from plastic (e.g., Delrin, polycarbonate, Nylon), metal, and/or other suitable materials.

In the illustrated embodiment, the implant handle 216 further includes an atrial-fixation release actuator 722, a cinch actuator 724, a clip actuator 726, and a clip release actuator 728 (collectively “actuators 722-728”). The actuators 722-728 can be coupled to the connector 720, the housing 723, and/or one another. In the illustrated embodiment, the atrial-fixation release actuator 722, the cinch actuator 724, and the clip actuator 726 comprise knobs that are rotatable about a longitudinal axis L (FIG. 7B) of the implant handle 216, and the clip release actuator 728 comprises a pull member that is movable (e.g., proximally) along and/or in a direction parallel to the longitudinal axis L. In other embodiments, the actuators 722-728 can comprise buttons, levers, sliders, and/or other actuation members.

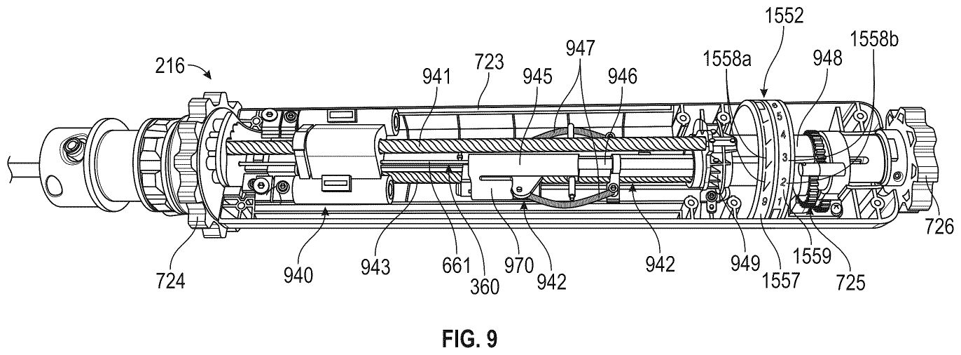

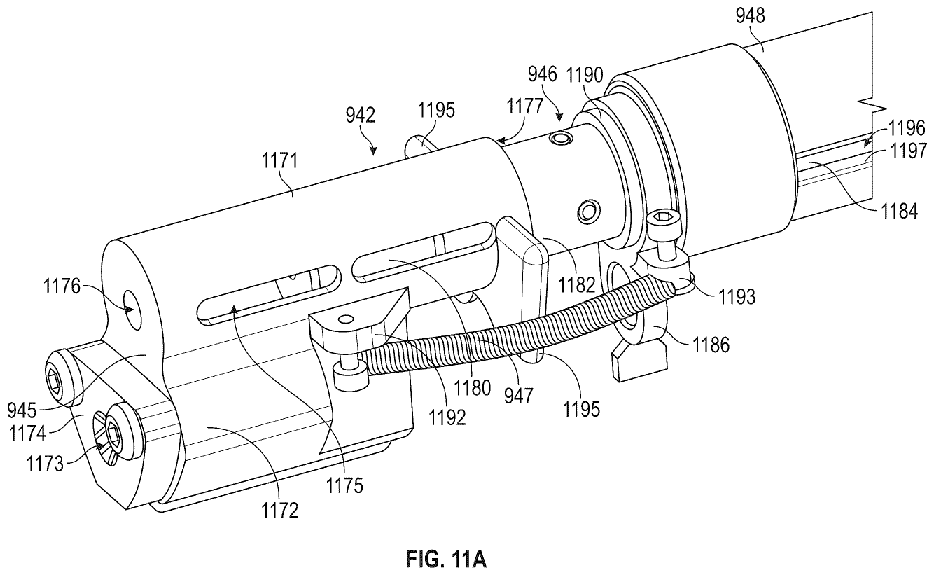

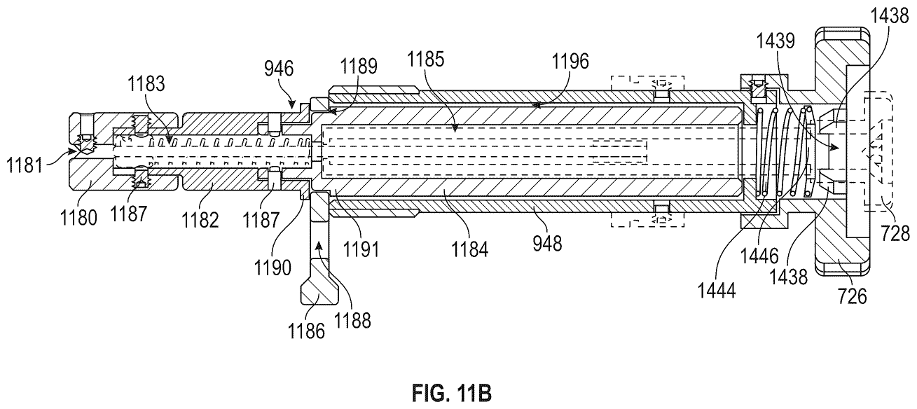

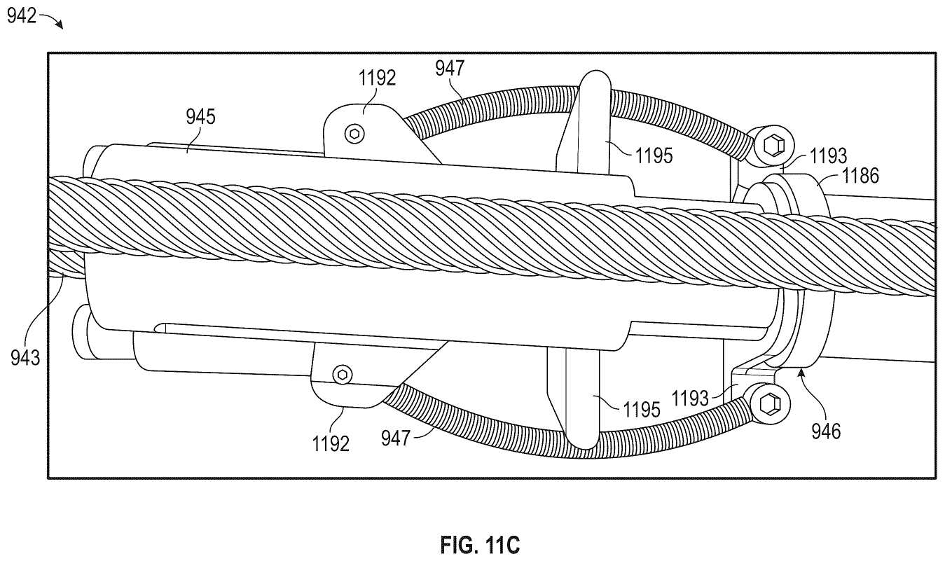

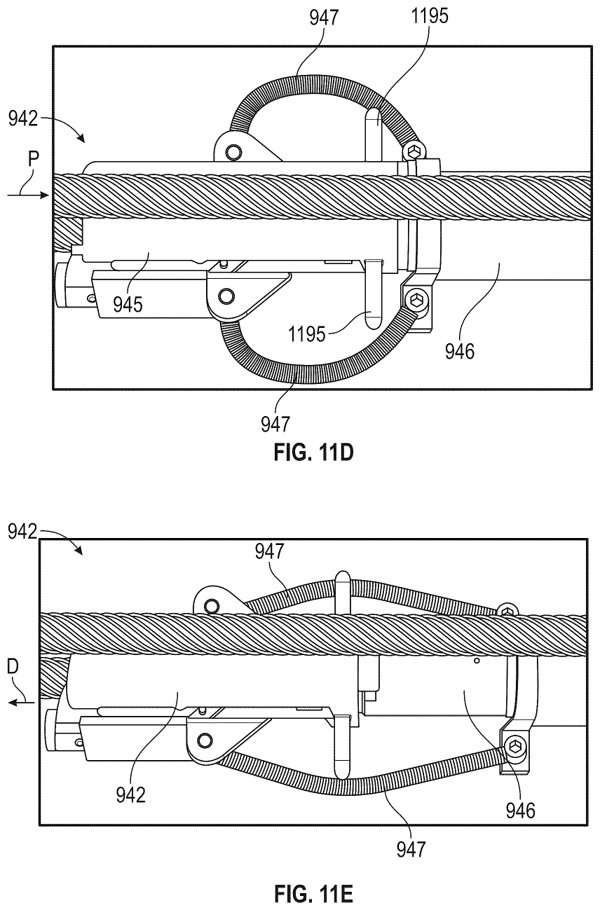

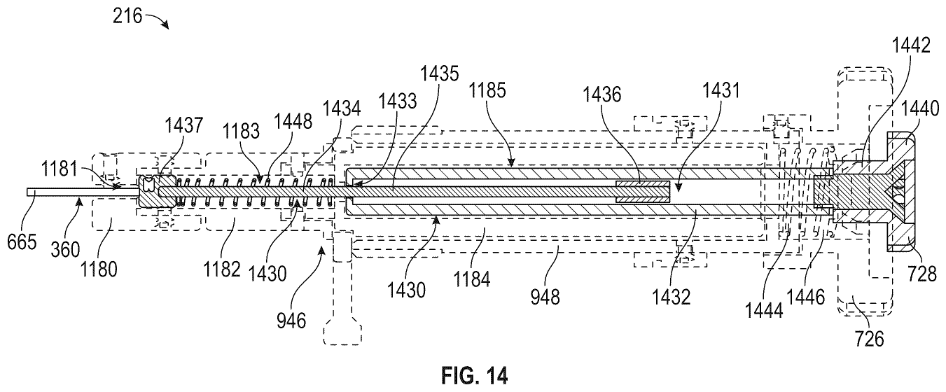

Referring to FIGS. 2-7B, the actuators 722-728 are configured to be manipulated/actuated by a user to (i) release the atrial-fixation member 382 of the implantable device 280 from the hub assembly 330, (ii) open/close the clip assembly 381, (iii) release the clip cable assembly 360 from the clip assembly 381, and (iv) cinch/uncinch the implantable device 280. More specifically, the atrial-fixation release actuator 722 can be operably coupled to the drive shaft 556 and can be actuated (e.g., rotated) to drive the drive shaft 556 to actuate the hub assembly 330 to release the atrial-fixation member 382 of the implantable device 280, as described in greater detail below with reference to FIG. 8. The cinch actuator 724 can be operably coupled to the first and second cinching lines 393, 394 and can be actuated (e.g., rotated) to increase/reduce a tension in the first and second cinching lines 393, 394 to cinch/uncinch the implantable device 280, as described in greater detail below with reference to FIGS. 9-10C and 12A-13C. In some embodiments, the cinch actuator 724 is further operably coupled to the clip cable assembly 360 such that actuation of the cinch actuator 724 simultaneously (e.g., during at least one overlapping time period) advances/retracts the clip cable assembly 360 to control an amount of slack in the clip cable assembly 360 during cinching/uncinching, as described in greater detail below with reference to FIGS. 9 and 11A-13C. The clip actuator 726 can be operably coupled to the clip cable assembly 360 and can be actuated (e.g., rotated) to drive the clip cable 661 to rotate the threaded member 674 to open/close the clip assembly 381, as described in greater detail below with reference to FIGS. 9 and 11A-11E. Finally, as described in greater detail below with reference to FIGS. 11B and 14, the clip release actuator 728 can be operably coupled to the lockwire 665 of the clip cable assembly 360 and can be actuated (e.g., pulled) to withdraw the lockwire 665 from the lock member 664 to decouple the clip cable assembly 360 from the clip assembly 381.

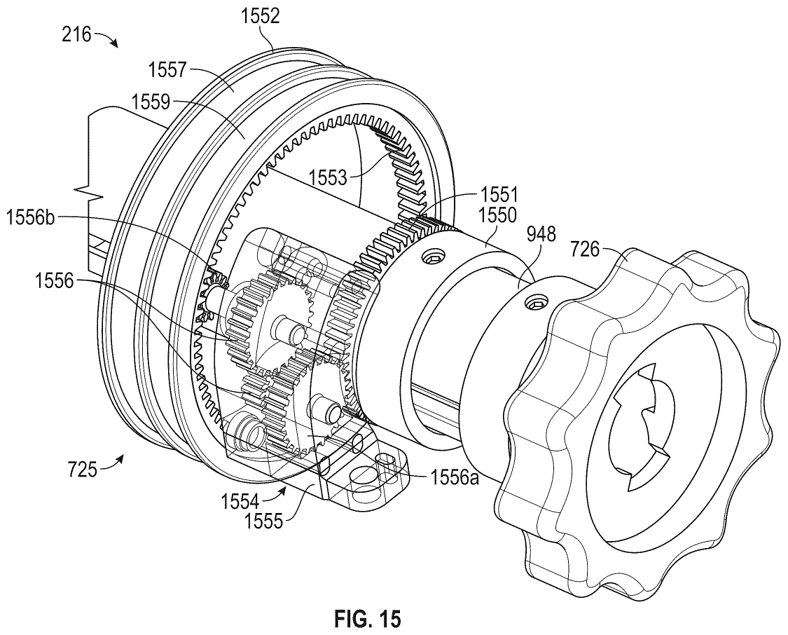

Referring to FIG. 7A, in some embodiments the implant handle 216 can include a clip position indicator assembly 725 operably coupled to the clip actuator 726 and that provides an indication of an amount the clip assembly 381 (FIGS. 3A, 6A, 6B, and 6E) is opened or closed. The clip position indicator assembly 725 is described in further detail below with reference to FIG. 15. The implant handle 216 can further include one or more markings on the housing 723 that indicate to a user the effect of a corresponding actuation of one or more of the actuators 722-728. For example, in the illustrated embodiment the implant handle 216 includes (i) a cinching indicator 727 that indicates that clockwise rotation of the cinch actuator 724 about the longitudinal axis L (FIG. 7B) uncinches the implantable device 280 (FIGS. 3A and 3B) and counterclockwise rotation of the cinch actuator 724 about the longitudinal axis L cinches the implantable device 280 and (ii) a clip indicator 729 that indicates that clockwise rotation of the clip actuator 726 about the longitudinal axis L (FIG. 7B) closes the clip assembly 381 (FIGS. 3A, 6A, 6B, and 6E) and counterclockwise rotation of the clip actuator 726 about the longitudinal axis L opens the clip assembly 381.

FIG. 8 is a partially cross-sectional isometric view of the connector 720 and the atrial-fixation release actuator 722 of the implant handle 216 of FIGS. 7A and 7B in accordance with embodiments of the present technology. The connector 720 is shown in cross-section in FIG. 8 for clarity. In the illustrated embodiment, the connector 720 includes/defines a series of interconnected lumens including a through lumen 832 and a flush lumen 833 extending generally orthogonal to the through lumen 832. The connector assembly 721 can comprise an implant shaft connector 834 positioned within the through lumen 832 and a flush port 835 positioned within the flush lumen 833. The implant shaft 206 (FIG. 7A) is configured to extend partially into the through lumen 832 and to be secured to the implant shaft connector 834. The flush port 835 can be fluidly connected to the lumen of the implant shaft 206 (FIG. 7A) to permit flushing of the lumen of the implant shaft 206. The connector assembly 721 can further comprise one or more adaptors, valves, sealing members, etc., that are configured to maintain hemostasis and/or facilitate the ingress/egress of fluids (e.g., blood, priming solution, etc.) into the implant shaft 206.

The drive shaft 556, the clip cable assembly 360 (FIGS. 3A and 6A-6E), and the first and second cinching lines 393, 394 (FIG. 3A) can extend proximally from the implant shaft 206 (FIG. 7A), through the implant shaft connector 834, to other components within the implant handle 216. For example, the connector 720 further comprises a drive shaft lumen 836 positioned to receive the drive shaft 556 through the implant shaft 206 (FIG. 7A). A proximal end portion of the drive shaft 556 is fixedly coupled to a gear 837 (e.g., a pinion gear). The gear 837 can be formed from metal (e.g., brass), plastic, and/or other suitably rigid materials. In some embodiments, the drive shaft 556 is secured to the gear 837 via a set screw and/or another suitable connection. In the illustrated embodiment, the atrial-fixation release actuator 722 is a ring gear having a plurality of teeth on an inner surface thereof configured (e.g., shaped, sized) to mate with corresponding teeth on an outer surface of the gear 837. Accordingly, the atrial-fixation release actuator 722 and the gear 837 form a gear assembly, and the atrial-fixation release actuator 722 is rotatable to rotate the gear 837 to rotate the drive shaft 556. As described in detail above with reference to FIGS. 3A-5C, rotation of the atrial-fixation release actuator 722 can rotate the drive shaft 556 to rotate the leadscrew 535 of the hub assembly 330 to drive the first housing member 533 of the inner hub component 432 distally relative to the second housing member 532 and the outer hub component 431 to the extended position in which the connectors 385 of the implantable device 280 can flex outward from the recesses 539—thereby releasing the atrial-fixation member 382 from the hub assembly 330 and the implant shaft 206.

In some embodiments, the atrial-fixation release actuator 722 includes a plurality of gripping features 838 around a perimeter thereof to facilitate easy gripping and rotation of the atrial-fixation release actuator 722. The gripping features 838 can be shaped in such a way that indicates to the user via tactile feedback the preferred direction of rotation. In some embodiments, the implant handle 216 includes one or more friction inducing features (e.g., compressed O-rings) and/or gear locks (not shown) that provide a frictional drag on the atrial-fixation release actuator 722 and/or the gear 837 that inhibits rotation of the atrial-fixation release actuator 722 to avoid rotation caused by inadvertent contact. Such friction inducing features can avoid accidental rotation and promote only purposeful actuation of the atrial-fixation release actuator 722.

FIG. 9 is a perspective top view of the implant handle 216 of FIGS. 7A and 7B in accordance with embodiments of the present technology. A portion (e.g., a top half) of the housing 723 is removed in FIG. 9 to illustrate the internal components of the implant handle 216. In the illustrated embodiment, the implant handle 216 includes a cinch mount assembly 940 coupled to the first and second cinching lines 393, 394 (not shown; FIG. 3A) and a clip cable mount assembly 942 coupled to the clip cable 661 of the clip cable assembly 360. The clip cable mount assembly 942 can include a clip cable carriage 945 coupled to a telescoping assembly 946 via a pair of biasing members 947, and the telescoping assembly 946 can be rotatably coupled to the clip actuator 726 via a rotatable sleeve 948 secured to the clip actuator 726. The cinch mount assembly 940 is slidably coupled to a first leadscrew 941 extending between the cinch actuator 724 and a support 949 coupled to the housing 723. Similarly, the clip cable mount assembly 942 (e.g., the clip cable carriage 945) is slidably coupled to a second leadscrew 943 extending between the cinch actuator 724 and the support 949. The first leadscrew 941 can be positioned above the second leadscrew 943. The clip position indicator assembly 725 is operably coupled to the clip actuator 726 via the sleeve 948.

FIG. 10A is a partially-transparent isometric view of the cinch mount assembly 940 of the implant handle 216 of FIG. 9 in accordance with embodiments of the present technology. In the illustrated embodiment, the cinch mount assembly 940 includes a cinch carriage 1050 (shown as partially transparent for clarity) having a middle portion 1051, a first side portion 1052a extending from middle portion 1051, and a second side portion 1052b extending from the middle portion 1051 opposite the first side portion 1052a. The cinch carriage 1050 further includes a proximal side portion 1064a and a distal side portion 1064b opposite the proximal side portion 1064a. The middle portion 1051 can define a first lumen 1053 for receiving the first leadscrew 941 (FIG. 9) therethrough. In some embodiments, an engagement member 1054 is positioned at least partially within the first lumen 1053 and defines a second lumen 1055 having a threaded surface configured to engage/mate with the first leadscrew 941. In other embodiments, the first lumen 1053 can be threaded and the engagement member 1054 can be omitted.

In the illustrated embodiment, the first side portion 1052a defines/includes a first tensioning lumen 1056a and the second side portion 1052b defines a second tensioning lumen 1056b. The cinch mount assembly 940 can further include a first shuttle member 1057a secured at least partially within the first tensioning lumen 1056a and a second shuttle member 1057b secured at least partially within the second tensioning lumen 1056b. The first and second shuttle members 1057a-b can each include a cinch attachment portion 1058, a stop portion 1059, and a rod portion 1060 extending between and coupling the cinch attachment portion 1058 and the stop portion 1059. In the illustrated embodiment, the cinch attachment portions 1058 are positioned proximally outside the first and second tensioning lumens 1056a-b and each include a post 1061 and a screw 1062. The first cinching line 393 (not shown; FIG. 3A) can be wound around the post 1061 of the first shuttle member 1057a (e.g., one time, two times, ten times, more than ten times) and then secured to the cinch attachment portion 1058 via the screw 1062. Similarly, the second cinching line 394 (not shown; FIG. 3A) can be wound around the post 1061 of the second shuttle member 1057b and then secured to the cinch attachment portion 1058 via the screw 1062. The rod portion 1060 and the stop portion 1059 of the first shuttle member 1057a are at least partially slidably positioned within the first tensioning lumen 1056a, and the rod portion 1060 and the stop portion 1059 of the second shuttle member 1057b are at least partially slidably positioned within the second tensioning lumen 1056b.

A first biasing member 1065a (shown schematically; e.g., a spring, a compression spring) can extend around the rod portion 1060 of the first shuttle member 1057a within the first tensioning lumen 1056a and be operably coupled between the stop portion 1059 of the first shuttle member 1057a and the first side portion 1052a (e.g., a portion of the first side portion 1052a proximate to the distal side portion 1064b of the cinch carriage 1050). Similarly, a second biasing member 1065b (shown schematically) can extend around the rod portion 1060 of the second shuttle member 1057b within the second tensioning lumen 1056b and be operably coupled between the stop portion 1059 of the second shuttle member 1057b and the second side portion 1052b (e.g., a portion of the second side portion 1052b proximate to the distal side portion 1064b of the cinch carriage 1050). The first and second biasing members 1065a-b can bias the first and second shuttle members 1057a-b, respectively, proximally toward the cinch carriage 1050.

In some aspects of the present technology, the first and second biasing members 1065a-b are configured to spring-load the first and second cinching lines 393, 394 (FIG. 3A) coupled to the cinch attachment portions 1058 of the first and second shuttle members 1057a-b, respectively, such that tension is maintained on the first and second cinching lines 393, 394 during manipulation of the delivery system 200 (FIG. 2). For example, although the stop portions 1059 are shown positioned fully proximally within the first and second tensioning lumens 1056a-b with the cinch attachment portions 1058 abutting the distal side portion 1064b of the cinch carriage 1050 in FIG. 10A, during operation of the delivery system 200, the first and second cinching lines 393, 394 can pull the first and second shuttle members 1057a-b at least partially distally through the first and second tensioning lumens 1056a-b against the biasing force of the first and second biasing members 1065a-b such that the first and second biasing members 1065a-b are loaded. The first and second biasing members 1065a-b can then drive the first and second shuttle members 1057a-b, respectively, proximally in response to any slack in the first and second cinching lines 393, 394 to maintain tension on the first and second cinching lines 393, 394 during, for example, any transient or gradual compression of the implant shaft 206 (FIG. 2) throughout the deployment procedure.

More specifically, FIGS. 10B and 10C are perspective side views of the cinch mount assembly 940 of FIG. 10A in accordance with embodiments of the present technology. Referring to FIG. 10B, the first shuttle member 1057a is shown in a spring-loaded position moved distally (e.g., via a force from the first cinching line 393 shown in FIG. 3A) away from the cinch carriage 1050 against the biasing force of the first biasing member 1065a (FIG. 10A). Similarly, referring to FIG. 10C, the second shuttle member 1057b is shown moved distally (e.g., via a force from the second cinching line 394 shown in FIG. 3A) away from the cinch carriage 1050 against the biasing force of the second biasing member 1065b (FIG. 10A).

Referring to FIG. 10A, in some embodiments the cinch mount assembly 940 further includes a spacer 1063 positioned within the first tensioning lumen 1056a. The first biasing member 1065a can extend between the spacer 1063 and the stop portion 1059 of the first shuttle member 1057a to further compress the first biasing member 1065a and/or increase the spring activation force of the first cinching line 393 (FIG. 3A; e.g., an upper cinching line configured to cinch a superior portion of the atrial-fixation member 382) required to move the first shuttle member 1057a against the biasing force of the first biasing member 1065a. In some aspects of the present technology, this may be useful to control the shape and/or cinching dynamics of the atrial fixation member 382 (FIG. 3A) via different levels of tension in the first and second cinching lines 393, 394 (FIG. 3A). In some embodiments, the first biasing member 1065a is identical to the second biasing member 1065b such that the spacer 1063 increases the resistive force of the first biasing member 1065a relative to the second biasing member 1065b. In other embodiments, the first biasing member 1065a can be different (e.g., more resistive to compression) than the second biasing member 1065b to achieve a similar higher activation force while omitting the spacer 1063.

FIG. 11A is an enlarged isometric view of the clip cable mount assembly 942 and a portion of the rotatable sleeve 948 of FIG. 9 configured in accordance with embodiments of the present technology, FIG. 11B is a cross-sectional side view of the telescoping assembly 946, the sleeve 948, and the clip actuator 726, and FIGS. 11C-11E are enlarged top views of the clip cable mount assembly 942 of FIG. 11A. In the illustrated embodiment, the clip cable carriage 945 has an upper portion 1171 and a lower portion 1172. The lower portion 1172 can define a drive lumen 1173 (partially obscured) for receiving the second leadscrew 943 (FIG. 9) therethrough. In some embodiments, an engagement member 1174 is coupled to the clip cable carriage 945, at least partially defines the drive lumen 1173, and has a threaded surface configured to engage/mate with the second leadscrew 943. In other embodiments, the drive lumen 1173 can be threaded within the lower portion 1172 and the engagement member 1174 can be omitted.

In the illustrated embodiment, the upper portion 1171 defines a clip cable lumen 1175 having a proximal opening 1176 and a distal opening 1177. The proximal opening 1176 can be configured (e.g., sized and shaped) to slidably receive the clip cable assembly 360 (FIG. 9) therethrough, and the distal opening 1177 can be configured to slidably receive a portion of the telescoping assembly 946 therethrough. In some embodiments, the distal opening 1177 has a larger dimension (e.g., diameter) than the proximal opening 1176.

As shown in FIGS. 11A and 11B, the telescoping assembly 946 can include (i) a clip cable mount 1180 defining a first lumen 1181 (FIG. 11B), (ii) a first elongated (e.g., tubular) member 1182 coupled to the clip cable mount 1180 and defining a second lumen 1183 (FIG. 11B), (iii) a second elongated (e.g., tubular) member 1184 coupled to the first elongated member 1182 and defining a third lumen 1185 (FIG. 11B), and (iv) a coupling member 1186 extending around the second elongated member 1184. The clip cable 661 (FIG. 9) can be secured to the clip cable mount 1180, and the lockwire 665 (FIGS. 6A-6C and 6E) can extend through the first lumen 1181 to various clip release components (shown as partially transparent in FIG. 11B) positioned within the second lumen 1183 and/or the third lumen 1185 and operably coupled to the clip release actuator 728 (FIG. 11B), as described in greater detail below with reference to FIG. 14. The clip cable mount 1180, the first elongated member 1182, and the second elongated member 1184 can be fixedly secured together via, for example, fasteners 1187 (FIG. 11B; e.g., screws) such that these components can move (e.g., rotate, translate) together. The coupling member 1186 can define a first aperture 1188 for receiving the second leadscrew 943 (FIG. 9) therethrough and a second aperture 1189 for receiving the second elongated member 1184 therethrough.

In some embodiments, the coupling member 1186 is fixed in position longitudinally relative to the clip cable mount 1180, the first elongated member 1182, and the second elongated member 1184, but rotatably mounted thereto such that these components can rotate relative to the coupling member 1186. For example, in the illustrated embodiment, the second aperture 1189 of the coupling member 1186 is rotatably coupled to/over the second elongated member 1184 and longitudinally fixed in position between a first flange 1190 of the first elongated member 1182 and a second flange 1191 (FIG. 11B) of the second elongated member 1184.

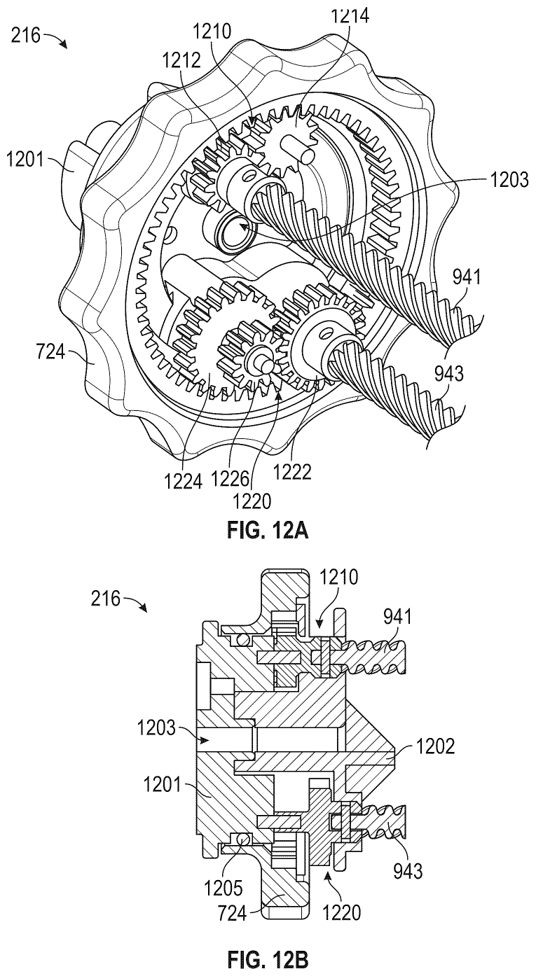

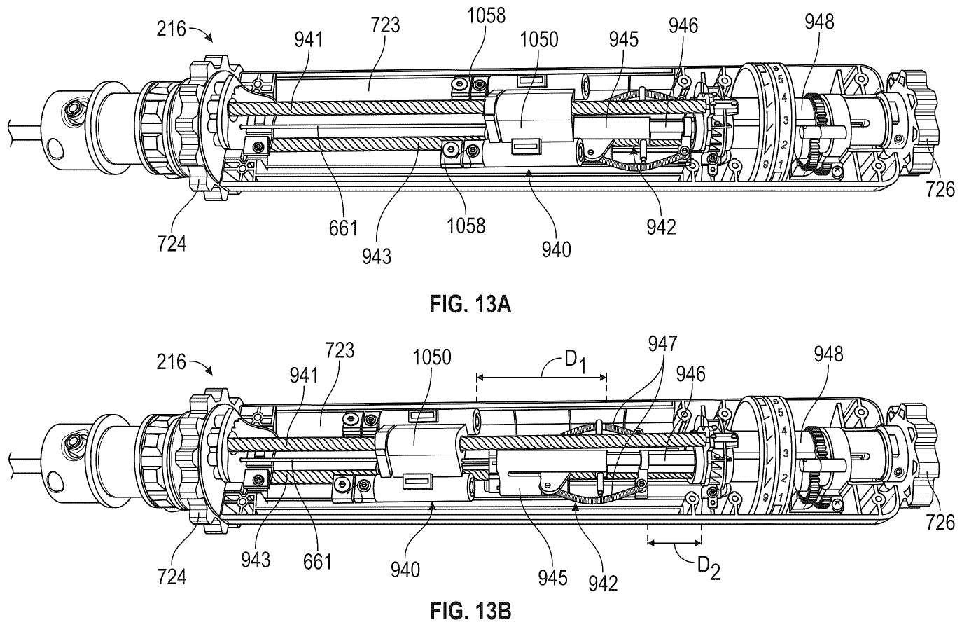

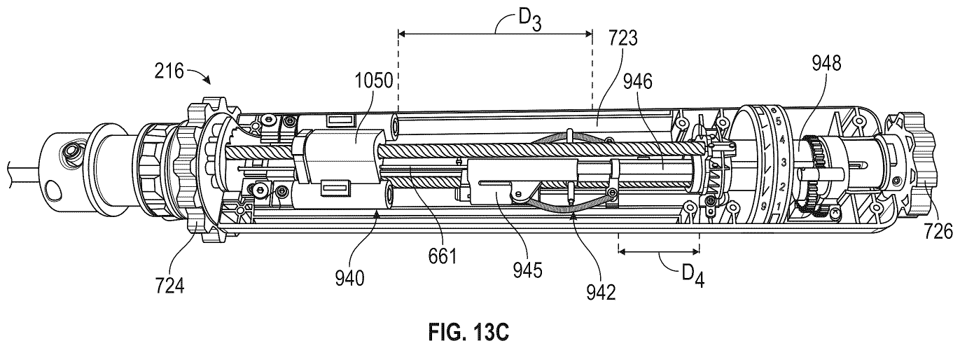

Referring to FIGS. 11A and 11C, the clip cable carriage 945 includes a pair of first spring mounts 1192 (one of the first spring mounts 1192 is obscured in FIG. 11A) extending from opposite side portions thereof, and the coupling member 1186 similarly includes a pair of second spring mounts 1193 (one of the second spring mounts 1193 is obscured in FIG. 11A) extending from opposite side portions thereof. The biasing members 947 (one of the biasing members 947 is obscured in FIG. 11A) are coupled between corresponding ones of the first and second spring mounts 1192, 1193. The biasing members 947 can be extension springs and can operably couple the clip cable carriage 945 to the telescoping assembly 946 such that the clip cable carriage 945 and the telescoping assembly 946 can translate together. More specifically, as described in further detail below with reference to FIGS. 12A-13C, the second leadscrew 943 can be rotated (e.g., via the cinch actuator 724 shown in FIGS. 7 and 9) to drive the clip cable carriage 945 along the second leadscrew 943 (FIG. 11C) via the engagement of the second leadscrew 943 with the drive lumen 1173, and the biasing members 947 can correspondingly drive the telescoping assembly 946 to move with the clip cable carriage 945.

In the illustrated embodiment, the clip cable carriage 945 further includes a pair of extension portions 1195 extending from opposite side portions thereof proximal of the first spring mounts 1192. Referring to FIG. 11C, the extension portions 1195 are configured (e.g., shaped, sized, positioned) to contact the biasing members 947 to bow the biasing members 947 outward away from the longitudinal axis L (FIG. 7B) of the implant handle 216. In some aspects of the present technology, the biasing members 947 “float” the telescoping assembly 946 relative to the clip cable carriage 945 to inhibit over tensioning of the clip cable 661 (FIG. 9) when the clip cable carriage 945 is driven proximally or distally by the second leadscrew 943 (FIG. 9). In some aspects of the present technology, this can help limit excessive tensile forces on the clip cable 661 and distal attachment features.

As shown in FIG. 11D, if the clip cable carriage 945 is driven proximally (as indicated by arrow P) toward the telescoping assembly 946 with more force (e.g., at a greater rate) than desired, the biasing members 947 can bow outwardly away from the extension portions 1195 such that not all of the linear movement of the clip cable carriage 945 is transmitted to the telescoping assembly 946 (and the clip cable 661 attached thereto as shown in FIG. 9). For example, referring to FIG. 11A, the clip cable mount 1180 and the first elongated member 1182 can move distally relative to and telescope within the clip cable lumen 1175 of the clip cable carriage 945 as the biasing members 947 bow outwardly—thereby decreasing a distance between the clip cable carriage 945 and the coupling member 1186 and inhibiting over tensioning of the clip cable 661 (FIG. 9). Likewise, referring to FIG. 11E, if the clip cable carriage 945 is driven distally as indicated by arrow D away from the telescoping assembly 946 with excessive force, the biasing members 947 can stretch to absorb the excessive force such that not all of the linear movement of the clip cable carriage 945 is transmitted to the telescoping assembly 946 (and the clip cable 661 attached thereto as shown in FIG. 9). More specifically, referring to FIG. 11A, the clip cable mount 1180 and the first elongated member 1182 can move proximally relative to and telescope within and/or out of the clip cable lumen 1175 of the clip cable carriage 945 as the biasing members 947 stretch-thereby increasing a distance between the clip cable carriage 945 and the coupling member 1186 and inhibiting over tensioning of the clip cable 661 (FIG. 9). Accordingly, the force applied to the clip cable 661 via the clip cable carriage 945 can be controlled as the clip cable carriage 945 moves both proximally and distally.

Referring again to FIGS. 11A and 11B, the sleeve 948 defines a telescoping lumen 1196 configured (e.g., shaped, sized) to slidably receive the second elongated member 1184 of the telescoping assembly 946. The second elongated member 1184 can move (e.g., telescope) proximally and distally within the telescoping lumen 1196 as the clip cable carriage 945 drives the telescoping assembly 946. Moreover, the second elongated member 1184 and the sleeve 948 can be configured (e.g., shaped, sized) such that rotation of the sleeve 948 via rotation of the clip actuator 726 rotates the second elongated member 1184 to rotate the telescoping assembly 946 to rotate the clip cable 661 (FIG. 9). For example, the sleeve 948 can include/define one or more slots 1197 (FIG. 11A) that receive corresponding projections (not shown; e.g., fins) of the second elongated member 1184 in a “keyed” arrangement. Alternatively or additionally, the second elongated member 1184 can have a cross-sectional shape configured to mate with a corresponding cross-sectional shape of an inner surface of the sleeve 948 in a non-rotational arrangement. For example, the second elongated member 1184 and the inner surface of the sleeve 948 can each have a polygonal (e.g., triangular, rectangular, square, hexagonal) shape. Accordingly, referring to FIGS. 9, 11A, and 11B, the clip cable 661 is rotationally coupled to the clip actuator 726 but remains free to slide (e.g., telescope) within the implant handle 216 (FIGS. 7A and 7B) via the force-controlled coupling of the telescoping assembly 946 to the clip cable carriage 945.

FIGS. 12A and 12B are a proximally-facing isometric view and a cross-sectional side view, respectively, of the implant handle 216 of FIGS. 7A and 7B including the cinch actuator 724, the first leadscrew 941, and the second leadscrew 943 in accordance with embodiments of the present technology. In the illustrated embodiment, the implant handle 216 includes a first connector 1201 coupling the cinch actuator 724 to proximal components of the implant handle 216 (e.g., the connector 720, the atrial-fixation release actuator 722, etc., shown in FIGS. 7A and 7B) and a second connector 1202 (omitted in FIG. 12A for clarity) coupling the cinch actuator 724 to distal components of the implant handle 216 (e.g., the housing 723 shown in FIGS. 7A and 7B). The first and second connectors 1201, 1202 can collectively define a lumen 1203 for receiving and routing the clip cable assembly 360 (FIG. 9) therethrough. In the illustrated embodiment, the first leadscrew 941 is operably (e.g., rotatably) coupled to the cinch actuator 724 via a first gear assembly 1210, and the second leadscrew 943 is operably coupled to the cinch actuator 724 via second gear assembly 1220. The cinch actuator 724 can be a ring gear having a plurality of teeth on an inner surface thereof configured (e.g., shaped, sized) to mate with corresponding teeth of the first and second gear assemblies 1210, 1220 such that rotation of the cinch actuator 724 rotates the first and second leadscrews 941, 943 via rotation of the first and second gear assemblies 1210, 1220, respectively.

More specifically, referring to FIG. 12A, the first gear assembly 1210 can include a first gear 1212 mounted to the first leadscrew 941 and a second gear 1214 mounted to the first connector 1201. The first gear 1212 is spaced apart and does not engage the cinch actuator 724, and the second gear 1214 is positioned between and engages (e.g., via mating teeth) both the cinch actuator 724 and the first gear 1212. Accordingly, rotation of the cinch actuator 724 rotates the second gear 1214, which transmits the rotation to the first gear 1212 and the first leadscrew 941 fixed thereto. In the illustrated embodiment, the second gear assembly 1220 includes a first gear 1222 mounted to the second leadscrew 943, a second gear 1224 mounted to the first connector 1201, and a third gear 1226 mounted to the second gear 1224. The first gear 1222 is spaced apart and does not engage the cinch actuator 724, the second gear 1224 engages (e.g., via mating teeth) the cinch actuator 724, and the third gear 1226 is fixed to rotate with the second gear 1224 and engages (e.g., via mating teeth) the first gear 1222. Accordingly, rotation of the cinch actuator 724 rotates the second gear 1224 and the third gear 1226 fixed thereto, and the third gear 1226 transmits the rotation to the first gear 1222 and the second leadscrew 943 fixed thereto. The gears of the first and second gear assemblies 1210, 1220 can comprise plastic (e.g., nylon), metal (e.g., brass), and/or other suitably rigid materials. In some embodiments, the implant handle 216 includes an O-ring 1205 (FIG. 12B) between the cinch actuator 724 and the first connector 1201 that provides a frictional drag on the cinch actuator 724. Such frictional drag can inhibit rotation of the cinch actuator 724 to avoid rotation caused by inadvertent contact and/or forces transmitted from the implantable device 280 (FIGS. 2-3A), such as tension on the cinch mount assembly 940 (FIG. 9) from the first and second cinching lines 393, 394 (FIG. 3A).

In some aspects of the present technology, the first and second gear assemblies 1210, 1220 are configured to differentially transmit the rotation of the cinch actuator 724 to the first and second leadscrews 941, 943. For example, the gears of the first and second gear assemblies 1210, 1220 can be sized to transmit a selected amount of rotation of the cinch actuator 724 to the first and second leadscrews 941, 943 in a ratio other than 1:1. In some embodiments, the first and second gear assemblies 1210, 1220 are configured such that rotation of the cinch actuator 724 rotates the first leadscrew 941 at a faster rotational rate than the second leadscrew 943. For example, rotation of the cinch actuator 724 can rotate the first leadscrew 941 between about 2.0-4.0, between about 2.5-3.5, about 3 times, and/or about 2.8 times faster than the second leadscrew 943. Such a differential rotation rate can correspondingly drive the cinch mount assembly 940 (FIG. 9) and the clip cable mount assembly 942 (FIG. 9) along the first and second leadscrews 941, 943, respectively, at different rates.

FIGS. 13A-13C are perspective top views of the implant handle 216 of FIGS. 7A and 7B in a cinched position, a partially-cinched position, and an uncinched position, respectively, in accordance with embodiments of the present technology. A portion (e.g., a top half) of the housing 723 is removed in FIG. 9 to illustrate the internal components of the implant handle 216. Referring to FIG. 13A, in the cinched position, the cinch mount assembly 940 is positioned proximally within the housing 723 along the first leadscrew 941 such that the first and second cinching lines 393, 394 (FIG. 3A) coupled to the cinch attachment portions 1058 are drawn proximally through the implant handle 216 and the implant shaft 206 (FIG. 7A) to cinch the implantable device 280 (FIGS. 2-3A). The clip cable mount assembly 942 is likewise positioned proximally within the housing 723 along the second leadscrew 943. In some embodiments, the cinch mount assembly 940 is configured (e.g., shaped, sized) to ride over a portion of the clip cable mount assembly 942 in the cinched position to help reduce an overall length of the implant handle 216.

To uncinch the implantable device 280 (FIGS. 2-3A), a user can grip and rotate the cinch actuator 724 (e.g., in a clockwise direction) to rotate the first and second leadscrews 941, 943 via the first and second gear assemblies 1210, 1220 (FIGS. 12A and 12B) to drive the cinch mount assembly 940 and the clip cable mount assembly 942 distally along the first and second leadscrews 941, 943, respectively. For example, in the partially-cinched position shown in FIG. 13B, rotation of the cinch actuator 724 has driven the cinch mount assembly 940 distally through the housing 723 along the first leadscrew 941 a first distance D₁ from the cinched position via the engagement of the first leadscrew 941 with the threaded first lumen 1053 and/or second lumen 1055 (FIG. 10A) of the cinch carriage 1050. The distal movement of the cinch mount assembly 940 reduces the tension in the first and second cinching lines 393, 394 (FIG. 3A) to allow the implantable device 280 (FIGS. 2A-3B) to partially expand. Likewise, the cinch actuator 724 has simultaneously driven the clip cable mount assembly 942 distally through the housing 723 along the second leadscrew 943 a second distance D₂ from the cinched position via the engagement of the second leadscrew 943 with the drive lumen 1173 (FIG. 11A) of the clip cable carriage 945. As described in detail above with reference to FIGS. 11A-11E, the clip cable carriage 945 drives the telescoping assembly 946 and the coupled clip cable 661 distally via the biasing members 947. In some embodiments, the first distance D₁ is greater (e.g., about 3 times greater, about 2.8 times greater) than the second distance D₂ as, for example, the first and second gear assemblies 1210, 1220 (FIGS. 12A and 12B) differentially transmit the rotation of the cinch actuator 724 to rotation of the first and second leadscrews 941, 943, as described in detail above with reference to FIGS. 12A and 12B.

Similarly, in the uncinched position shown in FIG. 13C, rotation of the cinch actuator 724 has simultaneously driven (i) the cinch mount assembly 940 farther distally through the housing 723 along the first leadscrew 941 a third distance D₃ from the cinched position and (ii) clip cable mount assembly 942 distally through the housing 723 along the second leadscrew 943 a fourth distance D₄ from the cinched position. Again, the third distance D₃ can be greater than the fourth distance D₄ due to the differential transmission of torque from the cinch actuator 724 to the first and second leadscrews 941, 943. The distal movement of the cinch mount assembly 940 to the uncinched position further reduces the tension in the first and second cinching lines 393, 394 (FIG. 3A) to allow the implantable device 280 (FIGS. 2A-3B) to fully expand. Referring to FIGS. 13A-13B together, the cinch actuator 724 can be rotated in an opposite direction to drive the cinch mount assembly 940 and the clip cable mount assembly 942 proximally within the housing 723 from the uncinched position to the cinched position.

Accordingly, referring to FIGS. 3A, 3C, and 13A-13C together, the implant handle 216 couples the movement (e.g., actuation) of the cinch mount assembly 940 to cinch/uncinch the implantable device 280 to corresponding movement of the clip cable mount assembly 942. In some aspects of the present technology, the coupled movement of the clip cable mount assembly 942 can act to provide a predefined amount of slack (e.g., optimized tension) of the clip cable 661 throughout cinching/uncinching of the implantable device 280. For example, the clip assembly 381 is positioned laterally away from the implant shaft 206 in the uncinched position (FIG. 3A) and is more aligned with the longitudinal axis of the implant shaft 206 in the cinched position (FIG. 3C) such that the clip cable assembly 360 extends along a longer path distal of the delivery shaft 204 in the uncinched position. Accordingly, the coupled movement of the clip cable mount assembly 942 and the cinch mount assembly 940 acts to move the clip cable 661 distally during uncinching of the implantable device 280 to maintain slack in in the clip cable 661, inhibit excessive tension in the clip cable 661, inhibit the clip cable 661 from pulling the implantable device 280 and inhibiting expansion thereof, etc. Likewise, the coupled movement of the clip cable mount assembly 942 and the cinch mount assembly 940 acts to move the clip cable 661 proximally during cinching of the implantable device 280 to inhibit excessive slack in in the clip cable 661, inhibit kinking of the clip cable 661, inhibit the clip cable 661 from exerting pressure on the implantable device 280, etc. In some embodiments, the ratio of the rotation rates of the first and second leadscrews 941, 943 and the corresponding axial travel lengths of the cinch mount assembly 940 and the clip cable mount assembly 942 can be selected based on the geometry of the implantable device 280 to optimize the tension of the clip cable 661 during cinching/uncinching. For example, as noted above, in some embodiments the ratio of the rotation rate of the first leadscrew 941 and/or the axial travel length of the cinch mount assembly 940 to the rotation rate of the second leadscrew 943 and/or the axial travel length of the clip cable mount assembly 942 is between about 2-4:1, between about 2.5-3.5, about 3:1, or about 2.8:1. The ratio can be selected based on the geometry of the implantable device 280.

Referring to FIGS. 13A-13C, the telescoping assembly 946 translates (e.g., telescopes) within the sleeve 948, as described in detail above with reference to FIGS. 11A-11B, when the clip cable mount assembly 942 is axially driven during cinching/uncinching. For example, the telescoping assembly 946 is positioned farther proximally within the sleeve 948 in the cinched position as shown in FIG. 13A, and translates progressively farther distally through the sleeve 948 during uncinching as shown in FIGS. 13B and 13C. Referring to FIGS. 3A, 6A-6E, 11A, 11B, and 13A-13C together, at any point during the cinching/uncinching of the implantable device 280, the clip actuator 726 can be rotated to rotate the sleeve 948 to rotate the second elongated member 1184 and the clip cable mount 1180 coupled thereto to thereby rotate the clip cable 661 and the threaded member 674 of the clip assembly 381 to open/close the clip assembly 381.

FIG. 14 is a cross-sectional side view of a lockwire release assembly 1430 of the implant handle 216 of FIGS. 7A and 7B positioned within the telescoping assembly 946 and the sleeve 948 in accordance with embodiments of the present technology. In the illustrated embodiment, the lockwire release assembly 1430 includes (i) a tubular member 1432 positioned within the third lumen 1185 of the second elongated member 1184 and defining a plunger lumen 1431, and (ii) a plunger 1434 slidably positioned within the plunger lumen 1431. More specifically, the plunger 1434 can comprise a rod 1435 extending through a distal opening 1433 of the tubular member 1432 and having a proximal portion coupled to a plunger head 1436 and a distal portion coupled to a lockwire connector 1437. The lockwire 665 of the clip cable assembly 360 can extend through the first lumen 1181 of the clip cable mount 1180 and be secured to the lockwire connector 1437. The lockwire connector 1437 can be slidably positioned within the telescoping assembly 946 within, for example, the second lumen 1183 of the first elongated member 1182.

The lockwire release assembly 1430 is in a locked position in FIG. 14 in which the lockwire 665 extends between the lock member 664 of the clip cable assembly 360 to secure the clip cable assembly 360 to the clip assembly 381, as described in detail above with reference to FIGS. 6A-6E. In some embodiments, a first biasing member 1448 (e.g., a compression spring) is positioned between a distal portion of the second elongated member 1184 and the lockwire connector 1437. Accordingly, the first biasing member 1448 can bias the lockwire connector 1437 distally within the second lumen 1183 to bias the lockwire 665 distally to inhibit or even prevent unintended retraction of the lockwire 665 that could lead to premature deployment/release of the clip assembly 381. In other embodiments, the lockwire release assembly 1430 can comprise a threaded feature or snap-fit feature on the lockwire 665 (e.g., a distal portion of the lockwire 665) instead of or in addition to the first biasing member 1448 to inhibit unintended retraction of the lockwire 665.

In the illustrated embodiment, the clip release actuator 728 is coupled to (e.g., fixed to) a proximal portion of the tubular member 1432. More specifically, the clip release actuator 728 can include a knob portion 1440 and a body portion 1442 extending from the knob portion 1440. The knob portion 1440 can be configured (e.g., shaped, sized) to be grasped and manipulated (e.g., rotated, pulled, depressed) by a user and is accessible at the proximal end portion of the implant handle 216 (FIGS. 7A and 7B). In some embodiments, the clip release actuator 728 is releasably coupled to the clip actuator 726, the sleeve 948, and/or another proximal component of the implant handle 216 (FIG. 9) in the locked position. For example, the body portion 1442 of the clip release actuator 728 can include one or more engagement features (not shown) that engage corresponding features of the clip actuator 726 in the locked position. More specifically, an inner surface of the clip actuator 726 can have one or more protrusions 1438 (FIG. 11B; obscured in FIG. 14) spaced apart by recesses 1439, and the body portion 1442 of the clip release actuator 728 can have corresponding protrusions (obscured in FIGS. 11B and 14) configured to be inserted through the recesses 1439 and positioned, via rotation of the knob portion 1440, distally behind the corresponding protrusions 1438. A second biasing member 1444 (e.g., a compression spring; also shown in FIG. 11B) can extend between a proximal portion of the sleeve 948 and a platform 1446 (also shown in FIG. 11B), such as a washer. In the locked position, the second biasing member 1444 biases the platform 1446 against a distal portion of the body portion 1442 of the clip release actuator 728 to maintain the engagement between the protrusions of the body portion 1442 and the corresponding protrusions 1438 (FIG. 11B) of the clip release actuator 728. To unlock the clip release actuator 728, the knob portion 1440 can be depressed distally against the biasing force of the second biasing member 1444 and rotated to align the protrusions of the body portion 1442 with corresponding ones of the recesses 1439 (FIG. 11B) and then pulled proximally to pull the body portion 1442 past the protrusions 1438 (FIG. 11B). In some embodiments, the second biasing member 1444 can help drive the clip release actuator 728 proximally after it has been rotated to align the protrusions with the recesses 1439.

Referring to FIGS. 13A-14, when the implant handle 216 is actuated to cinch/uncinch the implantable device 280 (FIGS. 2-3A), the lockwire 665 can translate with the telescoping assembly 946. For example, as the telescoping assembly 946 moves distally to uncinch the implantable device 280, the telescoping assembly 946 can pull the lockwire connector 1437 and attached lockwire 665 distally. During the distal movement, the plunger 1434 can move distally through the plunger lumen 1431 of the tubular member 1432, which is fixed in longitudinal position via the engagement of the clip release actuator 728 with the clip actuator 726. Likewise, as the telescoping assembly 946 moves proximally to cinch the implantable device 280, the telescoping assembly 946 can push the lockwire connector 1437 and attached lockwire 665 proximally. During the proximal movement, the plunger 1434 can move proximally through the plunger lumen 1431 of the tubular member 1432. Accordingly, as the telescoping assembly 946 maintains a desired (e.g., optimal) tension in the clip cable 661, the telescoping assembly 946 simultaneously moves the lockwire 665 to match the tension in the clip cable 661 and take up slack, introduce slack, etc.

Referring to FIGS. 6B and 14, to unlock the lockwire 665 from the clip assembly 381, the clip release actuator 728 can be unlocked from the clip actuator 726 as described in detail above, and then pulled proximally away from the clip actuator 726. The proximal movement of the clip release actuator 728 pulls the tubular member 1432 proximally through the third lumen 1185 of the second elongated member 1184 until the plunger head 1436 contacts a distal portion of the tubular member 1432 around the distal opening 1433. That is, the plunger head 1436 is configured (e.g., sized, shape) not to extend through the distal opening 1433. Further proximal movement of the clip release actuator 728 and the tubular member 1432 pulls the plunger 1434 proximally via the engagement of the plunger head 1436 with the tubular member 1432. As the plunger 1434 moves proximally, the lockwire connector 1437 moves proximally within the second lumen 1183 of the first elongated member 1182 to retract the lockwire 665 relative to the clip cable 661 and the clip assembly 381. The proximal movement of the lockwire 665 moves the lockwire 665 from between the arms 666 of the lock member 664 of the clip cable assembly 360 to permit the arms 666 to flex radially inward toward the longitudinal axis L out of the apertures 691 in the clip assembly 381 to release the clip cable assembly 360 from the clip assembly 381, as described in detail above with reference to FIGS. 6A-6E. Alternatively, the proximal movement of the lockwire 665 moves the lockwire out of the lumen 1780 extending through the head 1769 and out of the lumen 1770 extending through the lock member 1764 of the clip cable assembly 1760 to permit the lock member 1764 to disengage the head 1769—thereby releasing the clip cable assembly 1760 from the clip assembly 381, as described in detail above with reference to FIGS. 17A-17C. In some embodiments, further proximal movement of the clip release actuator 728 can compress the first biasing member 1448 sufficiently that the first biasing member 1448 exerts a proximal force against the second elongated member 1184 that pushes the telescoping assembly 946 proximally to, for example, retract the clip cable assembly 360 away from the clip assembly 381.

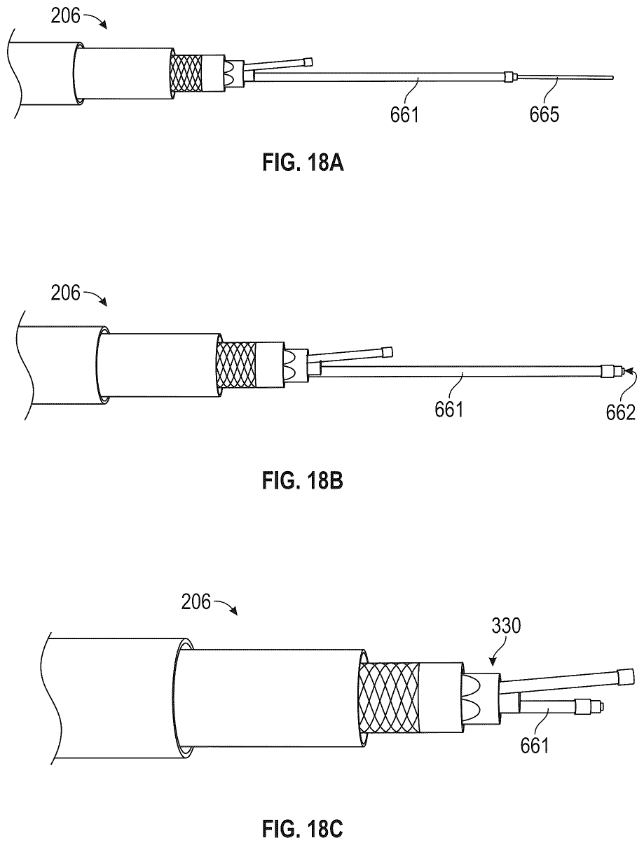

Accordingly, in some aspects of the present technology, proximal retraction of the clip release actuator 728 can (i) draw back the lockwire 665, (ii) unlock the clip cable assembly 360 (or the clip cable assembly 1760) from the clip assembly 381, and (iii) retract the clip cable assembly 360 away from the clip assembly 381. For example, FIGS. 18A-18C are enlarged side views of a distal portion of the implant shaft 206 during actuation of the clip release actuator 728 of FIG. 14 in accordance with embodiments of the present technology. Referring to FIGS. 14 and 18A, before retraction of the clip release actuator 728, the lockwire 665 extends through the clip cable 661 (e.g., to lock the clip cable 661 to clip assembly 381 as shown in FIGS. 6A, 6B, 17A, and 17C). Referring to FIGS. 14 and 18B, upon initial retraction of the clip release actuator 728, the lockwire 665 (FIG. 18A) is pulled proximally at least partially through the lumen 662 of the clip cable 661 to unlock the clip cable assembly 360 (or the clip cable assembly 1760) from the clip assembly 381 as described in detail with reference to FIGS. 6A-6E and 17A-17C. Finally, referring to FIGS. 14 and 18C, further retraction of the clip release actuator 728 can retract the clip cable 661 (e.g., proximally relative to the hub assembly 330) away from the clip assembly 381 (FIGS. 6A and 6B).

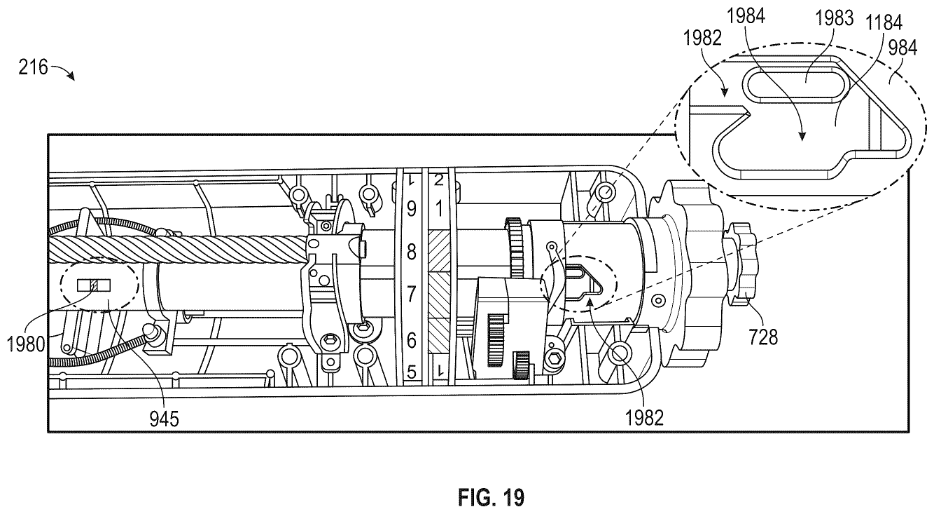

Referring to FIG. 14, in some embodiments the implant handle 216 includes one or more features for maintaining the position of the lockwire 665 and the clip release actuator 728 in a retracted position. FIG. 19, for example, is a perspective view of a portion of the implant handle 216 in accordance with additional embodiments of the present technology. In the illustrated embodiment, the implant handle 216 can comprise a lockwire lock feature 1980 configured to selectively lock the lockwire 665 (FIG. 14) and inhibit or even prevent distal movement of the lockwire 665 after retraction of the clip release actuator 728 by a predetermined amount. For example, the lockwire lock feature 1980 can comprise a latch or other automatic-locking feature positioned between the lockwire connector 1437 (FIG. 14) and the clip cable carriage 945, between the plunger 1434 (FIG. 14) and the clip cable carriage 945, and/or elsewhere in the implant handle 216. The lockwire lock feature 1980 can inhibit or even prevent the lockwire 665 from inadvertently springing back distally if the user lets go of the clip release actuator 728. Similarly, the implant handle 216 can include a clip cable lock feature 1982 configured to selectively lock the clip cable 661 (FIGS. 6A and 6B) and inhibit or even prevent distal movement of the clip cable 661 after retraction of the clip release actuator 728 by a further predetermined amount. In the illustrated embodiment, the clip cable lock feature 1982 comprises a cam lock slot including a protrusion or tab 1983 formed on an outer surface of the second elongated member 1184 and a locking slot 1984 formed through the sleeve 984. Upon retraction of the clip release actuator 728, the user can rotate the clip release actuator 728 to rotate the second elongated member 1184 to rotate the tab 1983 into the locking slot 1984 to lock the clip cable 661 (FIGS. 6A and 6B) in position. Accordingly, in some aspects of the present technology, in a single motion using only one knob (e.g., the clip release actuator 728) and no extra accessory tools, the user can retract the lockwire 665 and clip cable 661, then let go after the lockwire lock feature 1980 and the clip cable lock feature 1982 are engaged. This design simplifies the usability of the system during final release.

FIG. 15 is an isometric view of a proximal portion of the implant handle 216 of FIGS. 7A and 7B including the clip position indicator assembly 725 in accordance with embodiments of the present technology. The housing 723 is omitted in FIG. 15 for clarity. In the illustrated embodiment, the clip position indicator assembly 725 includes a drive ring 1550 fixed to an outer surface of the sleeve 948, an indicator ring 1552 positioned around the sleeve 948, and a gear assembly 1554 operably coupling the drive ring 1550 to the indicator ring 1552. The gear assembly 1554 can include a support 1555 (shown as partially transparent in FIG. 15 for clarity) and one or more gears 1556 (including an individually identified first gear 1556a and second gear 1556b) coupled to the support 1555. The support 1555 can be mounted to/within the housing 723 (FIGS. 7A and 7B). The drive ring 1550 can have a grooved portion 1551 along an outer surface thereof that is configured to engage the first gear 1556a. The indicator ring 1552 can have a grooved portion 1553 along an inner surface thereof that is configured to engage the second gear 1556b. When the clip actuator 726 is rotated to rotate the sleeve 948 to open/close the clip assembly 381 (FIGS. 3A, 3B, 6A, and 6B), the gear assembly 1554 can translate the rotation to corresponding rotation of the indicator ring 1552. More specifically, the grooved portion 1551 of the drive ring 1550 can engage and drive the first gear 1556a to rotate, and the gear assembly 1554 can translate the rotation to the indicator ring 1552 via the engagement of the second gear 1556b with the grooved portion 1553 of the indicator ring 1552.

The indicator ring 1552 can have various markings, numbers, and/or the like that provide an indication of an amount the clip assembly 381 is open/closed. In some embodiments, the indicator ring 1552 has a first indicator portion 1557 and a second indictor portion 1559 that can have the same or different types of indicators visible through different windows/apertures in the housing 723 (FIGS. 7A and 7B). For example, referring to FIG. 9, the first indicator portion 1557 of the indicator ring 1552 includes a plurality of first markings 1558a that directly illustrate a corresponding open/closed position of the clip assembly 381, and the second indicator portion 1559 of the indicator ring 1552 includes a plurality of second markings 1558b, such as numbers, that indicate an amount the clip assembly 381 is open/closed. Referring to FIGS. 9 and 15, the gears 1556 of the gear assembly 1554 can be configured (e.g., positioned, shaped, sized) such that the appropriate one of the first and second markings 1558a-b is positioned adjacent a corresponding window in the corresponding housing when the clip assembly 381 is in the indicated position.

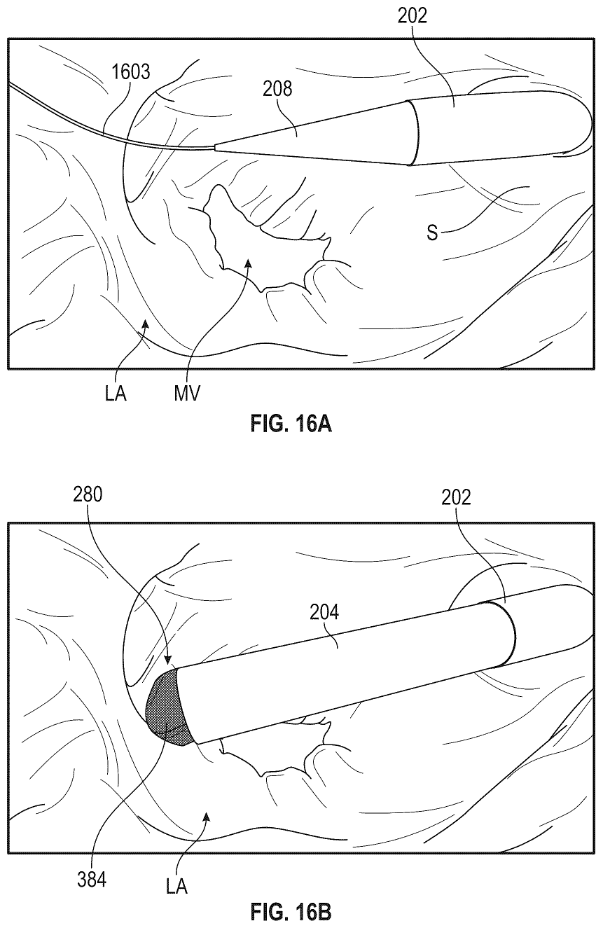

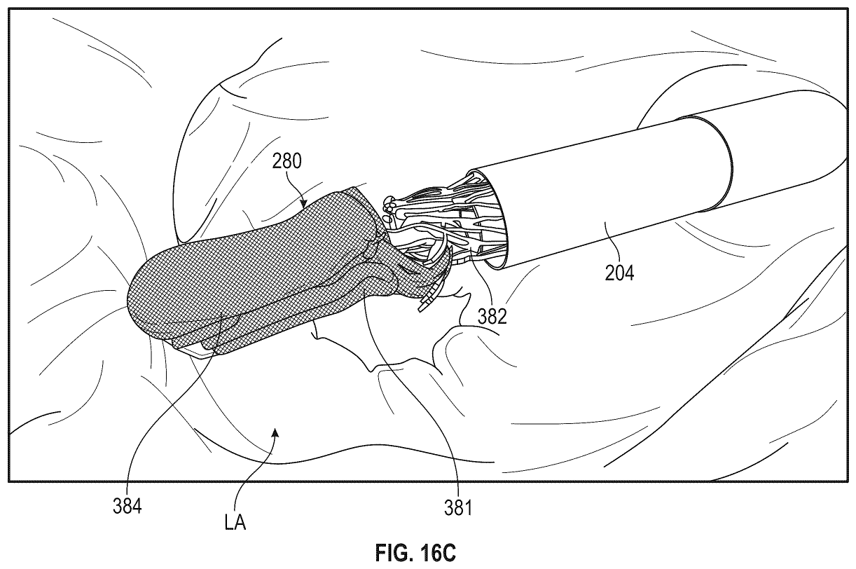

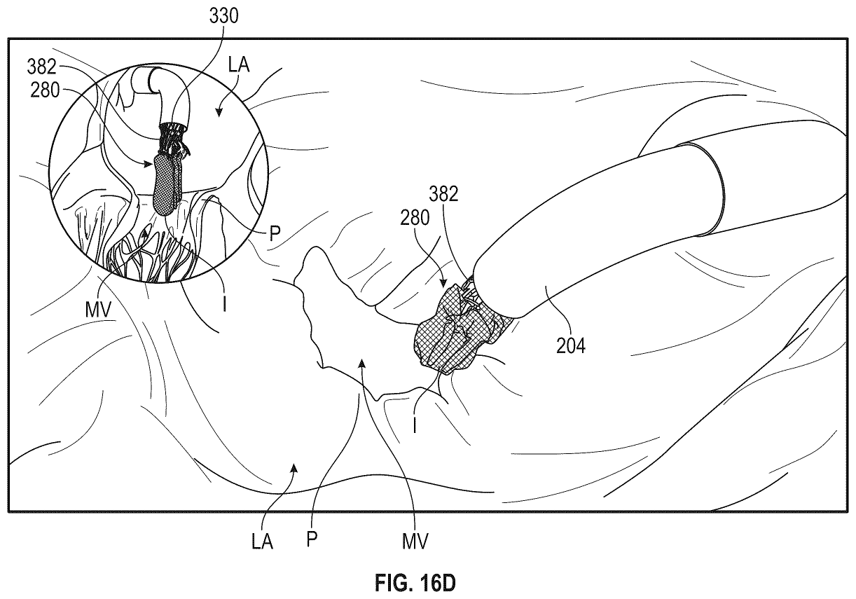

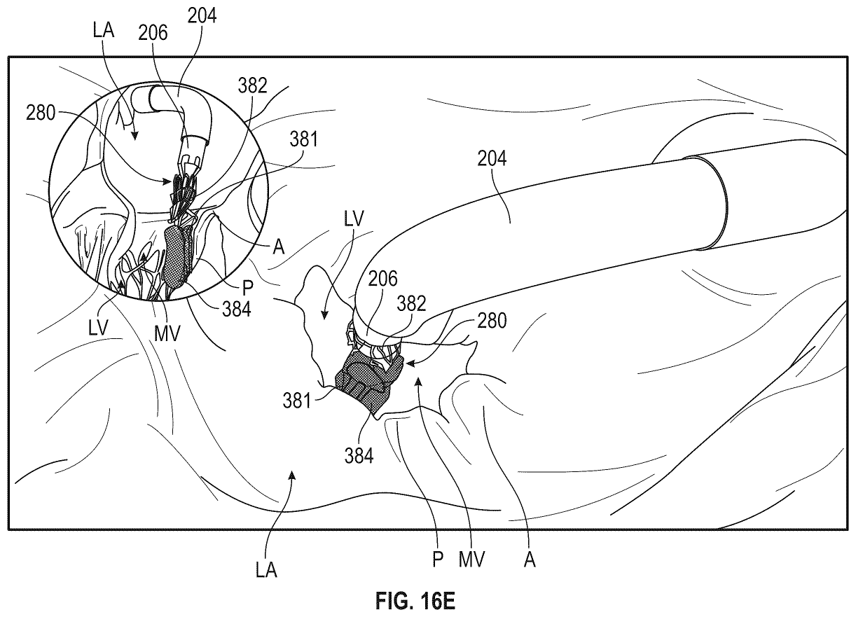

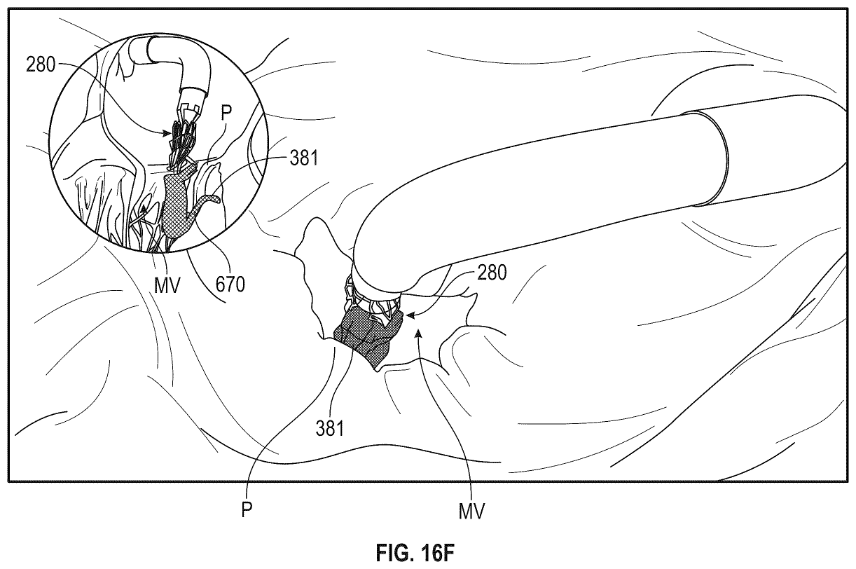

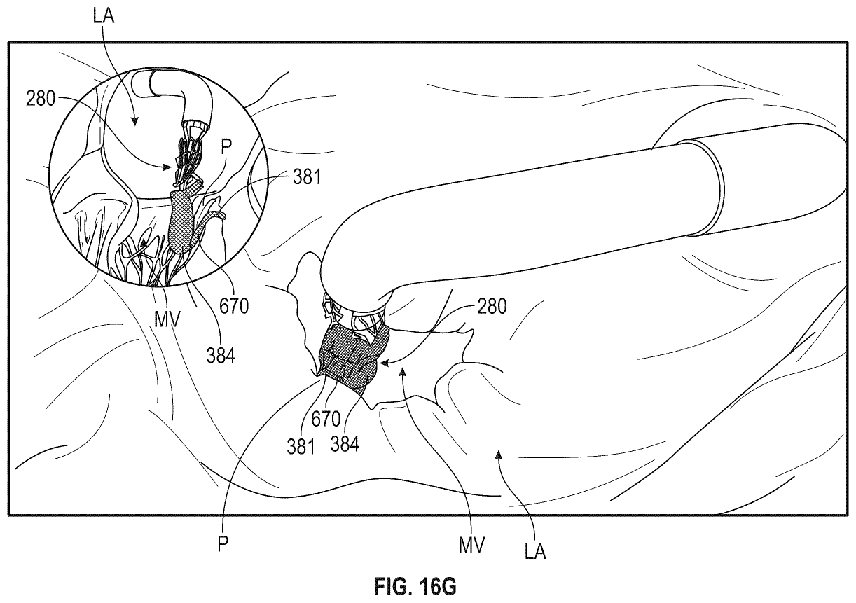

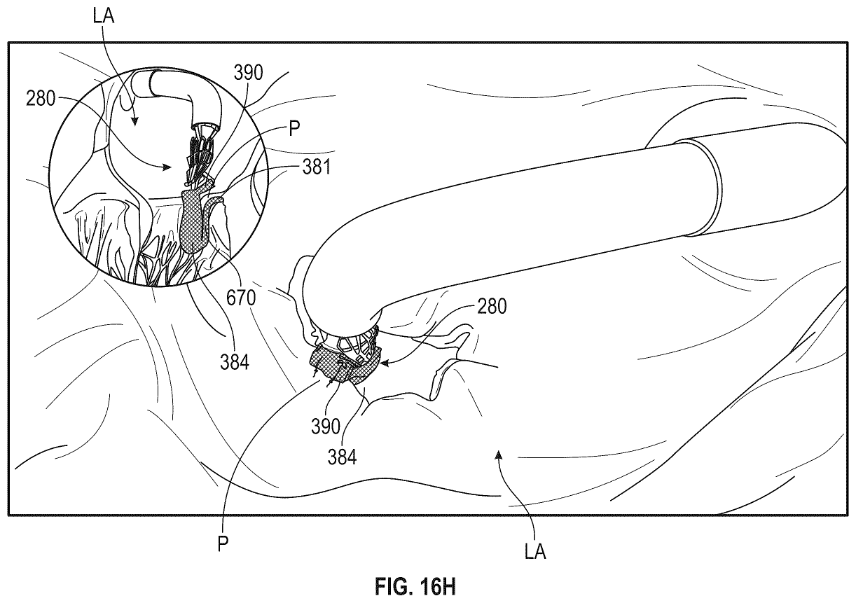

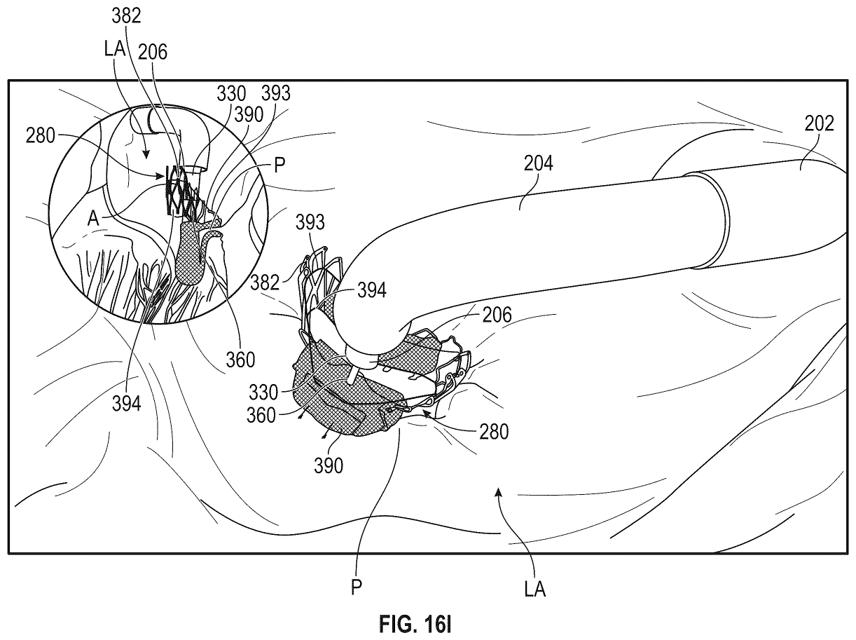

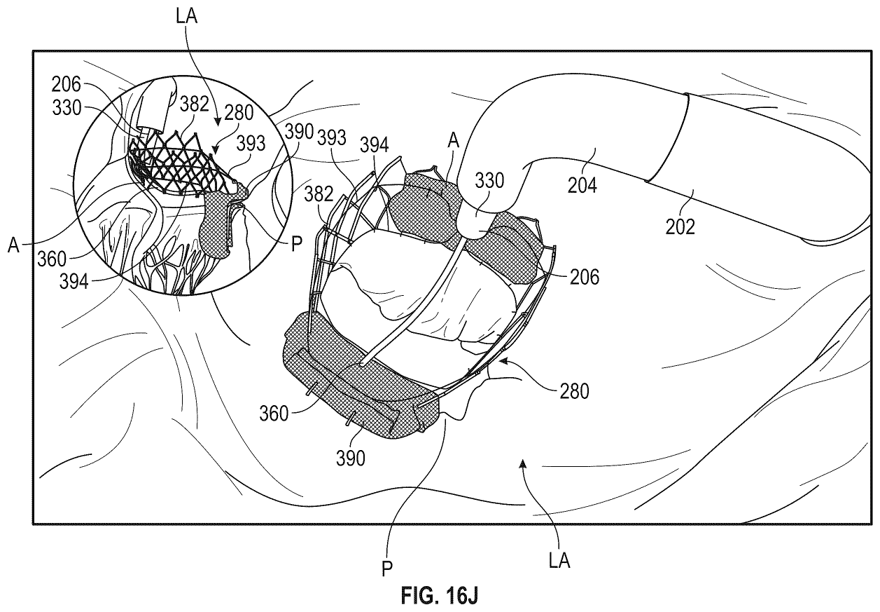

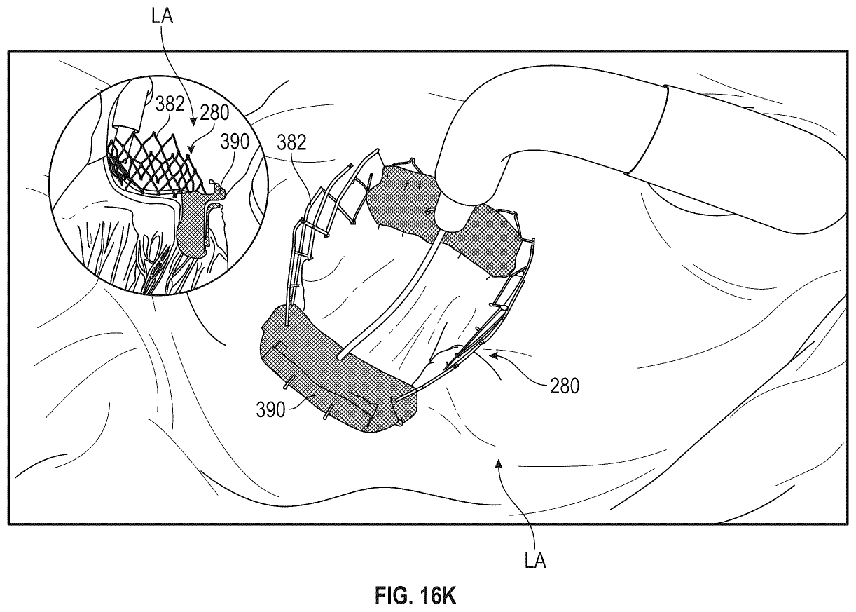

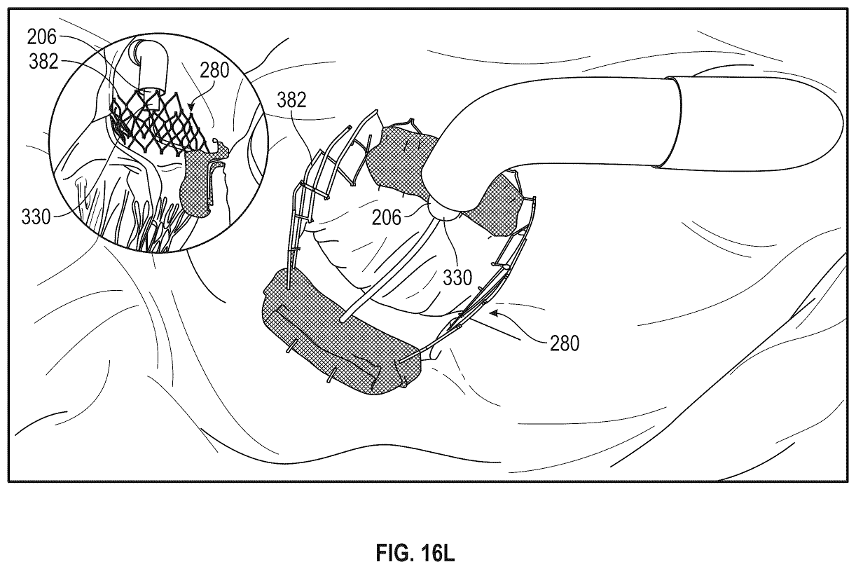

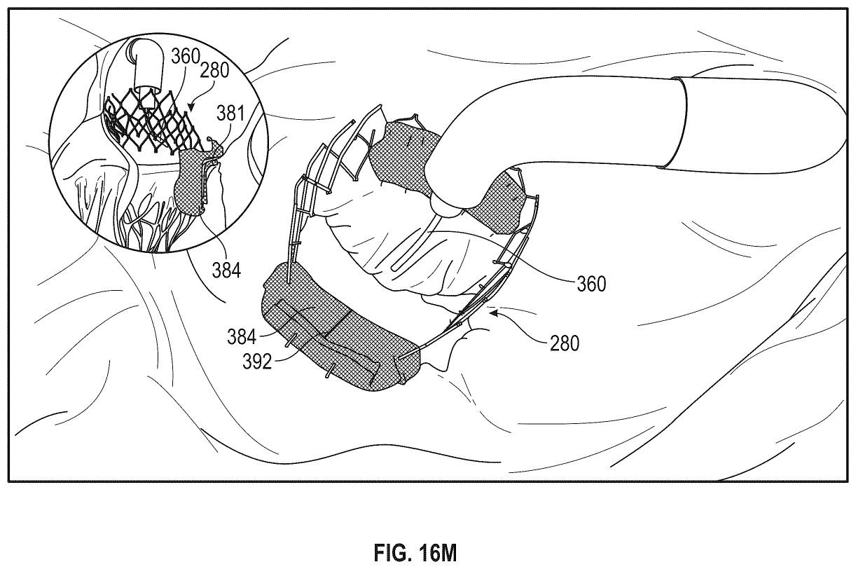

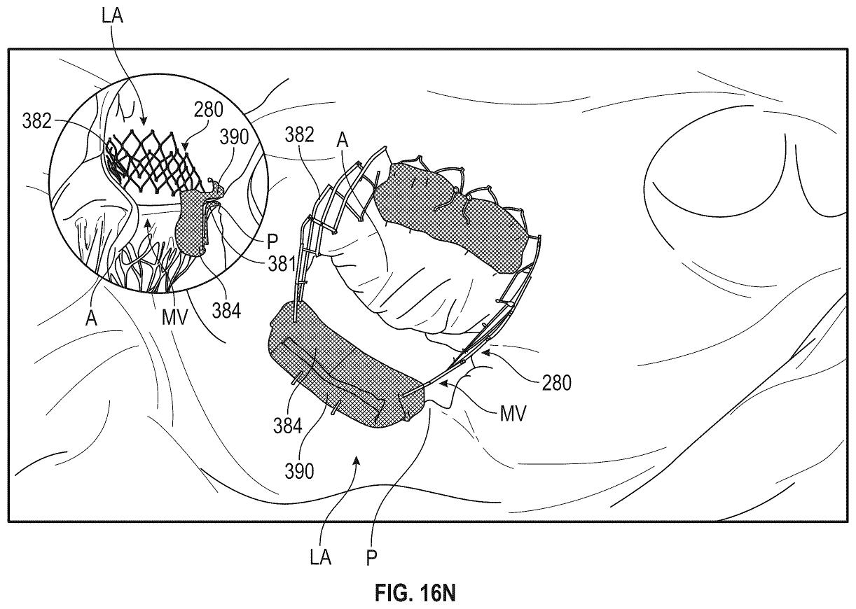

FIGS. 16A-16N illustrate various top and/or side views of the implantable device 280 and a distal portion of the delivery system 200 of FIGS. 2-15 during a method/procedure for delivering and deploying the implantable device 280 at a native mitral heart valve of a patient in accordance with embodiments of the present technology. Although the method is described in the context of delivering the implantable device 280 to a native mitral valve, the method can be used to endovascularly deliver and deploy the implantable device 280 and/or other implantable devices to other locations (e.g., to other cardiac valves) within a patient. Moreover, although the method is described in the context of the delivery system 200 described in detail above with reference to FIGS. 2-15, the illustrated method can be varied out using other suitable systems/components. In some embodiments, one or more of the stages of the method illustrated in FIGS. 16A-16N can be omitted, performed together, and/or performed in a different order.

Referring to the top view of FIG. 16A, the method can begin by positioning the guide catheter 202 within a left atrium LA of the patient. For example, the guide catheter 202 can be inserted with the dilator assembly 208 therein over a guidewire 1603 to traverse the venous system (e.g., via femoral or axillary access) to the right atrium and then across the interatrial septum S into the left atrium LA via a trans-septal approach (or through the atrial roof in a trans-atrial approach). In some embodiments, a distal portion of the guide catheter 202 can be positioned so that its distal-most end (e.g., the end furthest from the user) is in the left atrium LA. For example, the guide catheter 202 can extend to a location as shown in FIG. 16A, or the guide catheter 202 can be positioned farther in the left atrium LA to extend at least generally along a flow axis of a native mitral valve MV. This alignment can be achieved via a combination of torquing/steering the guide catheter 202 using the guide catheter handle 212, pre-shaping the end of the guide catheter 202, and/or flexing the guide catheter 202. However, in some embodiments, the guide catheter 202 may not have such complete steerability and its distal tip positioning may be more approximate such that the delivery catheter 204 can provide additional positioning at the desired location across the septum S in the left atrium LA.

Referring next to the top view of FIG. 16B, the method can continue by removing the guidewire 1603 and dilator assembly 208 (FIG. 16A) from the guide catheter 202 and advancing the delivery catheter 204 (including the implantable device 280 compressed therein) through the guide catheter 202 into the left atrium LA. In some embodiments, the coaptation member 384 at least partially extends out of the delivery catheter 204 during advancement through the guide catheter 202 to, for example, serve as an atraumatic end.

Referring to the top view of FIG. 16C, once positioned within the left atrium LA, the method can include at least partially unsheathing the implantable device 280 from the delivery catheter 204. For example, in the illustrated embodiment the coaptation member 384 is fully unsheathed with the clip assembly 381 in the closed position and the atrial-fixation member 382 is partially unsheathed. The implantable device 280 can be unsheathed by proximally retracting the delivery catheter 204 relative to the implantable device 280 and/or by distally advancing the implantable device 280 relative to the delivery catheter 204. More specifically, with additional reference to FIG. 2, the first actuation member 225a can be actuated to move the delivery catheter handle 214 proximally along the first leadscrew 226a to retract the delivery catheter 204 and/or the second actuation member 225b can be actuated to move the implant handle 216 distally along the second leadscrew 226b to advance the implant shaft 206 and the implantable device 280 coupled thereto.

Referring next to the top and side views of FIG. 16D, the method can include (i) steering the implantable device 280 within the left atrium LA toward the mitral valve MV of the patient, such that the inferior end portion I of the implantable device 280 is directed toward the mitral valve MV, and (ii) aligning the implantable device 280 with a portion of one or more desired native leaflets, such as a posterior leaflet P (e.g., a middle scallop (i.e., P2) of the posterior leaflet P). With additional reference to FIG. 2, the delivery actuation member 215 of the delivery catheter handle 214 can be actuated to deflect a distal portion of the delivery catheter 204 within the left atrium LA toward the mitral valve MV to axially align the implantable device 280 with the mitral valve MV, and/or the delivery catheter handle 214 can be rotated to steer the delivery catheter 204 through the left atrium LA toward the mitral valve MV to rotationally align the implantable device 280 with the posterior leaflet P. In some embodiments, the implantable device 280 is only partially-unsheathed from the delivery catheter 204 during this stage such that the delivery catheter 204 is positioned at least partially over the atrial-fixation member 382 and the hub assembly 330. In some aspects of the present technology, positioning the delivery catheter 204 at least partially over the implantable device 280 can aid in steering the implantable device 280 within the left atrium LA by providing additional rigidity, pushability, and/or torqueability to the implantable device 280 and/or limiting stiff length distal to the delivery catheter 204.

Referring next to the top and side views of FIG. 16E, the method can continue by advancing the implantable device 280 at least partially across the mitral valve MV. For example, the implant shaft 206 can be advanced (e.g., via actuation of the second actuation member 225b shown in FIG. 2) to move the implant shaft 206 and the implantable device 280 distally relative to the delivery catheter 204. Accordingly, advancing the implantable device 280 at least partially across the mitral valve MV can include fully unsheathing the implantable device 280 from the delivery catheter 204. In some embodiments, the implantable device 280 is fully unsheathed from the delivery catheter 204 before the implantable device 280 is advanced at least partially across the mitral valve MV. In some embodiments, the implantable device 280 is advanced to a target position in which (i) the atrial-fixation member 382 is positioned at least partially above an annulus A of the mitral valve MV in the left atrium LA and (ii) the coaptation member 384 is positioned at or below the annulus A in a left ventricle LV of the patient. In the illustrated embodiment, the clip assembly 381 of the implantable device 280 is in the closed position and is positioned proximate to the posterior leaflet P. In other embodiments, the clip assembly 381 can be adjusted to the open position (or an intermediate, partially-open position) before advancing the implantable device 280 across the mitral valve MV.

Referring next to the top and side views of FIG. 16F, the method can include opening the clip assembly 381 of the implantable device 280 such that the clip member 670 (obscured in the top view of FIG. 16F) of the clip assembly 381 such that the clip member 670 extends at least partially behind the posterior leaflet P. For example, with additional reference to FIGS. 6A-6E, 7A, 7B, 11A, and 11B, the clip actuator 726 of the implant handle 216 can be rotated to rotate the clip cable 661 of the clip cable assembly 360 to rotate the threaded member 674 of the clip assembly 381 to pivot the clip member 670 to the open position.

Referring to the top and side views of FIG. 16G, the method can then include retracting (e.g., via actuation of the second actuation member 225b shown in FIG. 2) the implantable device 280 proximally (e.g., raising the implantable device 280 toward the left atrium LA along a flow axis of the mitral valve MV) to position a portion (e.g., the middle scallop) of the posterior leaflet P between the coaptation member 384 and the clip member 670 of the clip assembly 381. Next, referring to the top and side views of FIG. 16H, the method can include closing the clip assembly 381 (obscured in the top view of FIG. 16H) to secure (e.g., capture, sandwich) the portion of the posterior leaflet P between the clip member 670 and the coaptation member 384. For example, with additional reference to FIGS. 6A-6E, 7A, 7B, 11A, and 11B, the clip actuator 726 of the implant handle 216 can be rotated to rotate the clip cable 661 of the clip cable assembly 360 to rotate the threaded member 674 of the clip assembly 381 to pivot the clip member 670 to the closed position. In the illustrated embodiment, the brim member 390 of the implantable device 280 is positioned above the posterior leaflet P in the left atrium LA.

Referring next to the top and side views of FIGS. 161 and 16J, the method can include uncinching the atrial-fixation member 382 of the implantable device 280 within the left atrium LA. FIG. 16I shows the atrial-fixation member 382 in a partially uncinched position, and FIG. 16J shows the atrial-fixation member 382 in a fully uncinched position. More specifically, with additional reference to FIGS. 13A-13C, the cinch actuator 724 can be rotated to advance the cinch mount assembly 940 distally within the housing to release tension from the first and second cinching lines 393, 394 to permit the atrial-fixation member 382 to expand within the left atrium LA. During uncinching, the hub assembly 330 of the implant shaft 206 can remain rigidly coupled to the anterior portion A of the atrial-fixation member 382 via the connectors 385 (FIG. 3C). Accordingly, in some embodiments the delivery catheter 204 and/or the implant shaft 206 can be steered away from the posterior leaflet P to permit the atrial-fixation member 382 to expand into contact with an anterior portion of the left atrium LA. In some embodiments, the implantable device 280 can alternatively or additionally be steered via the guide catheter 202. Alternatively or additionally, the expansion of the atrial-fixation member 382 can drive the implant shaft 206 and the delivery catheter 204 away from the posterior leaflet P toward the anterior portion of the left atrium LA. As described in detail above with reference to FIGS. 13A-14, as the atrial-fixation member 382 is uncinched and expands, the clip cable mount assembly 942 is driven distally with the cinch mount assembly 940 to maintain slack in in the clip cable assembly 360. As shown in FIG. 16J, after uncinching, the atrial-fixation member 382 can engage tissue of the anterior portion of the left atrium LA, and the brim member 390 can engage tissue of a posterior portion of the left atrium LA above the posterior leaflet P.

In some embodiments, after allowing the atrial-fixation member 382 to expand within the left atrium LA, the method can include determining/assessing whether the implantable device 280 is properly positioned and functioning properly. The performance of the implantable device 280, such as a reduction in an amount of mitral regurgitation, can be evaluated via transesophageal echocardiography (“TEE”) imaging and/or another suitable technique (e.g. radiopaque dye injection, real-time monitoring of left atrial pressure, etc). For example, the left atrial pressure can be monitored via a separate hemodynamic monitoring catheter and/or by attaching one or pressure transducers to a flush port of one or more of the catheters of the delivery system 200. If the implantable device 280 is not functioning properly, the implantable device can be repositioned or recovered and removed from the patient. For example, the first and second cinching lines 393, 394 can be cinched again (e.g., via rotation of the cinch actuator 724 of the implant handle 216 of FIGS. 7A and 7B) to radially compress the atrial-fixation member 382. Then, the clip assembly 381 can be opened (e.g., via the clip actuator 726 of the implant handle 216 of FIGS. 7A and 7B) to release the posterior leaflet P, and the implantable device 280 can be retracted into the left atrium LA. The implantable device 280 can then either be repositioned, or removed by, for example, retracting the implantable device 280 into the guide catheter 202

Referring next to the top and side views of FIG. 16K, if the implantable device 280 is properly positioned and functioning properly, the first and second cinching lines 393, 394 (FIGS. 16I and 16J) can be removed. For example, with additional reference to FIGS. 7A and 9, the cover 730 can be removed from over the opening 731 to provide access to the first and second cinching lines 393, 394 coupled to the cinch mount assembly 940. The first and second cinching lines 393, 394 can then be cut (or otherwise uncoupled from the cinch mount assembly 940) and removed through the opening 731. In some embodiments, removing the first and second cinching lines 393, 394 allows the atrial-fixation member 382 and/or the brim member 390 to further expand into engagement with tissue of the left atrium LA.

Referring to the top and side views of FIG. 16L, the method can include detaching the atrial-fixation member 382 of the implantable device 280 from the hub assembly 330 and the implant shaft 206. For example, as described in detail above with reference to FIGS. 3A-5C and 10, the atrial-fixation release actuator 722 can be rotated to rotate the drive shaft 556 to rotate the leadscrew 535 of the hub assembly 330 to drive the first housing member 533 of the inner hub component 432 distally relative to the second housing member 532 and the outer hub component 431 to the extended position in which the connectors 385 of the implantable device 280 can flex outward from the recesses 539—thereby releasing the atrial-fixation member 382 from the hub assembly 330 and the implant shaft 206.

Referring next to the top and side views of FIG. 16M, the method can include detaching the clip cable assembly 360 from the clip assembly 381 (obscured in top view of FIG. 16M) of the implantable device 280. For example, as described in detail above with reference to FIGS. 6B, 6E, and 14, the clip release actuator 728 of the implant handle 216 can be unlocked and then pulled proximally to (i) withdraw the lockwire 665 from within the lock member 664 of the clip cable assembly 360 to decouple the clip cable assembly 360 from the clip assembly 381 and (ii) withdraw the clip cable assembly 360 proximally from the clip assembly 381 (e.g., from out of the slit 392 in the coaptation member 384 shown in the top view of FIG. 16M).

Finally, referring to top and side views shown in FIG. 16N, the method can include withdrawing the delivery system 200 (FIG. 2; e.g., the guide catheter 202, the delivery catheter 204, and the implant shaft 206) from the patient. For example, the catheters 202-206 can be individually or collectively steered to a straight configuration and withdrawn from the patient such that only the implantable device 280 remains, as shown in FIG. 16M. Once deployed, the coaptation member 384 may extend in front of a portion (e.g., a central portion) of the posterior leaflet P (e.g., the P2 scallop of the posterior leaflet P) to position the coaptation member 384 in a location that allows it to coapt with an anterior leaflet A during systole (the anterior leaflet A is shown during diastole in FIG. 16N). The clip assembly 381 (obscured in the top view of FIG. 16N) is configured to extend behind and grasp one or more portions the posterior leaflet P to affix the one or more portions to the coaptation member 384. The atrial-fixation member 382 contacts an anterior portion of the left atrium LA, and the brim member 390 extends above the posterior leaflet P into the left atrium LA to contact tissue proximate thereto. The atrial-fixation member 382 extends in an anterior-superior direction from the coaptation member 384 into the left atrium LA to brace and fix the coaptation member 384 in position.

The following examples are illustrative of several embodiments of the present technology:
1. A system for repairing a cardiac valve, the system comprising:
a valve repair device comprising—
   a coaptation member configured to (a) coapt with at least a portion of a first native leaflet of the cardiac valve during systole and (b) displace at least a portion of a second native leaflet of the cardiac valve;
   a fixation member extending in a superior direction from the coaptation member, wherein the fixation member includes a superior end portion and an inferior end portion; and
   at least one connector extending radially inward from the fixation member from between the superior end portion and the inferior end portion; and
a delivery system configured to position the valve repair device at the cardiac valve, wherein the delivery system comprises—
   an implant shaft having a hub assembly configured to be releasably coupled to the at least one connector;
   a drive shaft operably coupled to the hub assembly; and
   a handle having an actuator operably coupled to the drive shaft, wherein the actuator is actuatable to drive the drive shaft to actuate the hub assembly to release the at least one connector from the hub assembly to decouple the valve repair device from the implant shaft.
2. The system of example 1 wherein the coaptation member and the fixation member together extend circumferentially about a vertical axis, and wherein the at least one connector is positioned at a first circumferential position relative to the vertical axis, and wherein the coaptation member is positioned at a second circumferential position relative to the vertical axis generally opposite to the first circumferential position.
3. The system of example 1 or example 2 wherein the at least one connector comprises a pair of connectors.
4. The system of example 3 wherein the actuator is actuatable to drive the drive shaft to actuate the hub assembly to simultaneously release the pair of connectors from the hub assembly to decouple the valve repair device from the implant shaft.
5. The system of any one of examples 1-4 wherein the hub assembly includes an outer hub component and inner hub component movable through the outer hub component, wherein the inner hub component includes an outer surface having at least one recess formed therein, wherein the at least one recess is shaped and sized to receive the at least one connector therein, wherein the hub assembly couples the implant shaft to the valve repair device when the at least one connector is positioned with the least one recess and the at least one recess is positioned within and covered by the outer hub component, and wherein the actuator is actuatable to drive the drive shaft to move the inner hub component through the outer hub component to uncover the at least one recess from the outer hub component to permit the at least one connector to move out of the at least one recess to decouple the valve repair device from the implant shaft.
6. The system of any one of examples 1-5 wherein the hub assembly includes an outer hub component and inner hub component movable through the outer hub component, and wherein the inner hub component has a non-circular shape defining a channel between the outer hub component and the inner hub component.

7. The system of example 6 wherein the valve repair device further includes a clip, wherein the delivery system further comprises a clip cable releasably operably coupled to the clip and configured to move the clip between an open position and a closed position, and wherein the clip cable extends through the channel between the outer hub component and the inner hub component.
8. A delivery system for endovascularly implanting a valve repair device at a cardiac valve, the delivery system comprising:
an implant shaft comprising a distal portion having a hub assembly, wherein the distal portion of the implant shaft is configured to be endovascularly delivered to an atrium, wherein the hub assembly is configured to be releasably coupled to at least one connector positioned between a superior end portion and an inferior end portion of an expandable fixation member of the valve repair device;
a drive shaft operably coupled to the hub assembly; and
a handle having an actuator operably coupled to the drive shaft, wherein the actuator is actuatable to drive the drive shaft to actuate the hub assembly to release the at least one connector from the hub assembly to decouple the valve repair device from the implant shaft.
9. The delivery system of example 8 wherein the valve repair device includes a coaptation member configured to (a) coapt with at least a portion of a first native leaflet of the cardiac valve during systole and (b) displace at least a portion of a second native leaflet of the cardiac valve, wherein the fixation member extends from the coaptation member, and wherein the at least one connector is positioned opposite the coaptation member relative to a vertical axis of the valve repair device.
10. The delivery system of example 8 or example 9, further comprising a delivery catheter, wherein the implant shaft extends through the delivery catheter, and wherein the hub assembly is configured to be releasably coupled to the at least one connector when the fixation member is at least partially expanded distal of the delivery catheter.
11. The delivery system of 10 wherein the valve repair device extends along a vertical axis, and wherein the hub assembly is configured to be releasably coupled to the at least one connector offset from the vertical axis when the fixation member is at least partially expanded distal of the delivery catheter.
12. A delivery system for endovascularly implanting a valve repair device at a cardiac valve, the delivery system comprising:
a clip cable configured to be releasably coupled to a clip assembly of the valve repair device;
a cinching line configured to extend at least partially around an expandable fixation member of the valve repair device and to cinch the fixation member to a compressed position; and
a handle having an actuator operably coupled to the clip cable and the cinching line, wherein the actuator is actuatable to simultaneously (a) reduce a tension in the cinching line to permit the fixation member to expand to an expanded position and (b) reduce a tension in the clip cable.
13. The delivery system of example 12 wherein the handle further comprises a clip cable mount assembly operably coupled to the clip cable and a cinch mount assembly operably coupled to the cinching line, wherein the actuator is actuatable to simultaneously move the clip cable mount assembly and the cinch mount assembly in a same direction to reduce the tension in the cinching line and reduce the tension in the clip cable.
14. The delivery system of example 13 wherein the actuator is actuatable to simultaneously move the clip cable mount assembly at a first rate and the cinch mount assembly at a second rate different than the first rate.
15. The delivery system of example 14 wherein the second rate is between about 2.5-3.5 times faster than the first rate.
16. The delivery system of any one of examples 13-15 wherein the clip cable mount assembly is movably mounted to a first lead screw, wherein the cinch mount assembly is movably mounted to a second lead screw, and wherein the actuator is rotatable to simultaneously (a) rotate the first lead screw to movably the clip cable mount assembly therealong to reduce the tension in the clip cable and (b) rotate the second lead screw to movably the cinch mount assembly therealong to reduce the tension in the cinching line.
17. The delivery system of example 16 wherein the actuator is rotatable to simultaneously rotate the first lead screw at a first rate and the second lead screw at a second rate different than the first rate.
18. The delivery system of example 17 wherein the second rate is between about 2.5-3.5 times faster than the first rate.
19. A handle of a delivery system for endovascularly implanting a valve repair device at a cardiac valve, the handle comprising:
a housing;
a first leadscrew carried by the housing;
a second leadscrew carried by the housing;
a cinch mount assembly operably coupled to the first leadscrew and configured to be coupled to a cinching line of the delivery system, wherein the cinching line is configured to extend at least partially around the valve repair device and to cinch the valve repair device to a compressed position;
a clip cable mount assembly operably coupled to the second leadscrew and configured to be coupled to a clip cable of the delivery system, wherein the clip cable is configured to be releasably coupled to a clip assembly of the valve repair device;
an actuator; and
a gear assembly operably coupling the actuator to the first and second leadscrews, wherein the actuator is actuatable to drive the gear assembly to simultaneously drive (a) the cinch mount assembly axially along the first leadscrew at a first rate and (b) the clip cable mount assembly axially along the second leadscrew at a second rate different than the first rate.
20 The handle of example 19 wherein the second rate is between about 2.5-3.5 times faster than the first rate.

21. A delivery system for endovascularly implanting a valve repair device at a cardiac valve, the delivery system comprising:
a clip cable configured to be releasably coupled to a clip assembly of the valve repair device; and
a handle having a first actuator and a second actuator operably coupled to the clip cable, wherein the first actuator is actuatable to drive the clip cable axially relative to the handle, and wherein the second actuator is actuatable to rotate the clip cable to open and close the clip assembly.
22. The delivery system of example 21 wherein the first actuator is actuatable to drive the clip cable axially relative to the handle to change a level of tension in the clip cable.
23. The delivery system of example 21 or example 22 wherein the handle further comprises a sleeve and an elongate member at least partially positioned within the sleeve, wherein the clip cable has a proximal portion fixedly coupled to the elongate member, wherein the first actuator is actuatable to move the elongate member at least partially through the sleeve, and wherein the second actuator is rotatable to rotate the sleeve to rotate the elongate member to rotate the clip cable to open and close the clip assembly.
24. The delivery system of example 23 wherein the handle further comprises:
a leadscrew;
a clip cable carriage movably mounted to the leadscrew; and
at least one spring operably mounted between the clip cable carriage and the elongate member, wherein the first actuator is actuatable to move the clip cable carriage along the leadscrew, and wherein the at least one spring is configured to transmit at least a portion of the movement of the clip cable carriage to the elongate member such that the movement of the clip cable carriage moves the elongate member to move the clip cable axially relative to the handle.
25. The delivery system of example 24 wherein the at least one spring comprises an extension spring.
26. The delivery system of example 24 or example 25 wherein the clip cable carriage further comprises at least one extension portion configured to bow the at least one spring outward away from a longitudinal axis of the handle.
27. The delivery system of any one of examples 24-26 wherein the at least one spring is configured to transmit less than all of the movement of the clip cable carriage to the elongate member when the first actuator is actuated to move the clip cable carriage along the leadscrew at above a predetermined rate.
28. A handle of a delivery system for endovascularly implanting a valve repair device at a cardiac valve, the handle comprising:
a housing;
a leadscrew positioned within the housing;
a clip cable mount assembly operably coupled to the leadscrew, wherein the clip cable mount assembly is configured to be coupled to a clip cable of the delivery system, and wherein the clip cable is configured to be releasably coupled to a clip assembly of the valve repair device;
a sleeve positioned around a portion of the clip cable mount assembly;
a first actuator operably coupled to the clip cable mount assembly, wherein the first actuator is actuatable to drive the clip cable mount assembly and the clip cable axially along the leadscrew, and wherein the portion of the clip cable mount assembly is configured to telescope within the sleeve as the clip cable mount assembly moves axially along the leadscrew; and
a second actuator operably coupled to the sleeve, wherein the second actuator is actuatable to rotate the sleeve such that the clip cable mount assembly rotates the clip cable to open and close the clip assembly.
29. The handle of example 28 wherein the sleeve includes an inner surface, wherein the portion of the clip cable mount assembly has an outer surface, and wherein the inner surface and the outer surface have a same cross-sectional shape.
30. The handle of example 28 or example 29 wherein the sleeve has at least one slot, wherein the portion of the clip cable mount assembly has an outer surface including at least one projection extending therefrom, and wherein the projection extends through the slot.
31. A method of implanting a valve repair device at a cardiac valve, the method comprising:
endovascularly delivering a distal portion of a delivery catheter to a chamber of a heart;
unsheathing at least a portion of the valve repair device from the delivery catheter while in the chamber of the heart;
advancing the valve repair device to a target position extending across the cardiac valve such that a fixation member of the valve repair device is positioned at a first side of the cardiac valve upstream of a native valve anulus of the cardiac valve and a coaptation member of the valve repair device is positioned at least partially at a second side of the cardiac valve proximate to native valve leaflets of the cardiac valve;
rotating, in a first direction, a clip cable coupled to a clip assembly extending from the coaptation member to open the clip assembly;
rotating, in a second direction, the clip cable to close the clip assembly to capture at least a portion of one of the native valve leaflets between the clip assembly and the coaptation member;
reducing a tension in a cinching line extending at least partially around the fixation member to allow the fixation member to at least partially expand; and
while reducing the tensioning in the cinching line, reducing a tension in the clip cable.
32. The method of example 31 wherein endovascularly delivering the distal portion of the delivery catheter to the chamber of the heart comprises advancing the distal portion with a portion of the coaptation member extending distally from the distal portion of the delivery catheter.
33 The method of example 32 wherein the portion of the coaptation member is atraumatic.
34. The method of any one of examples 31-33 wherein reducing the tension in the cinching line further comprises permitting an implant shaft releasably coupled to the fixation member to move away from a vertical axis of the valve repair device.
35. The method of example 34 wherein the fixation member extends in a superior direction from the coaptation member, wherein the fixation member includes a superior end portion and an inferior end portion, wherein the valve repair device further comprises at least one connector extending radially inward from the fixation member from between the superior end portion and the inferior end portion, and wherein the implant shaft has a hub assembly configured to be releasably coupled to the at least one connector.
36 The method of example 35 wherein the method further comprises, after reducing the tension in the cinching line, actuating a drive shaft operably coupled to the hub assembly to release the at least one connector from the hub assembly to decouple the valve repair device from the implant shaft.
37. The method of any one of examples 31-36 wherein the cardiac valve is a native mitral valve.

All numeric values are herein assumed to be modified by the term about whether or not explicitly indicated. The term about, in the context of numeric values, generally refers to a range of numbers that one of skill in the art would consider equivalent to the recited value (e.g., having the same function and/or result). For example, the term about can refer to the stated value plus or minus ten percent. For example, the use of the term about 100 can refer to a range of from 90 to 110, inclusive. In instances in which the context requires otherwise and/or relative terminology is used in reference to something that does not include, or is not related to, a numerical value, the terms are given their ordinary meaning to one skilled in the art.

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments can perform steps in a different order. The various embodiments described herein can also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms can also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications can be made without deviating from the technology. Further, while advantages associated with some embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

I/We claim:

1. A system for repairing a cardiac valve, the system comprising:
a valve repair device comprising—
a coaptation member configured to (a) coapt with at least a portion of a first native leaflet of the cardiac valve during systole and (b) displace at least a portion of a second native leaflet of the cardiac valve;
a fixation member extending in a superior direction from the coaptation member, wherein the fixation member includes a superior end portion and an inferior end portion; and
at least one connector extending radially inward from the fixation member from between the superior end portion and the inferior end portion; and
a delivery system configured to position the valve repair device at the cardiac valve, wherein the delivery system comprises—
an implant shaft having a hub assembly configured to be releasably coupled to the at least one connector;
a drive shaft operably coupled to the hub assembly; and
a handle having an actuator operably coupled to the drive shaft, wherein the actuator is actuatable to drive the drive shaft to actuate the hub assembly to release the at least one connector from the hub assembly to decouple the valve repair device from the implant shaft.

2. The system of claim 1 wherein the coaptation member and the fixation member together extend circumferentially about a vertical axis, and wherein the at least one connector is positioned at a first circumferential position relative to the vertical axis, and wherein the coaptation member is positioned at a second circumferential position relative to the vertical axis generally opposite to the first circumferential position.

3. The system of claim 1 wherein the at least one connector comprises a pair of connectors.

4. The system of claim 3 wherein the actuator is actuatable to drive the drive shaft to actuate the hub assembly to simultaneously release the pair of connectors from the hub assembly to decouple the valve repair device from the implant shaft.

5. The system of claim 1 wherein the hub assembly includes an outer hub component and inner hub component movable through the outer hub component, wherein the inner hub component includes an outer surface having at least one recess formed therein, wherein the at least one recess is shaped and sized to receive the at least one connector therein, wherein the hub assembly couples the implant shaft to the valve repair device when the at least one connector is positioned with the least one recess and the at least one recess is positioned within and covered by the outer hub component, and wherein the actuator is actuatable to drive the drive shaft to move the inner hub component through the outer hub component to uncover the at least one recess from the outer hub component to permit the at least one connector to move out of the at least one recess to decouple the valve repair device from the implant shaft.

6. The system of claim 1 wherein the hub assembly includes an outer hub component and inner hub component movable through the outer hub component, and wherein the inner hub component has a non-circular shape defining a channel between the outer hub component and the inner hub component.

7. The system of claim 6 wherein the valve repair device further includes a clip, wherein the delivery system further comprises a clip cable releasably operably coupled to the clip and configured to move the clip between an open position and a closed position, and wherein the clip cable extends through the channel between the outer hub component and the inner hub component.

8. A delivery system for endovascularly implanting a valve repair device at a cardiac valve, the delivery system comprising:
an implant shaft comprising a distal portion having a hub assembly, wherein the distal portion of the implant shaft is configured to be endovascularly delivered to an atrium, wherein the hub assembly is configured to be releasably coupled to at least one connector positioned between a superior end portion and an inferior end portion of an expandable fixation member of the valve repair device;
a drive shaft operably coupled to the hub assembly; and
a handle having an actuator operably coupled to the drive shaft, wherein the actuator is actuatable to drive the drive shaft to actuate the hub assembly to release the at least one connector from the hub assembly to decouple the valve repair device from the implant shaft.

9. The delivery system of claim 8 wherein the valve repair device includes a coaptation member configured to (a) coapt with at least a portion of a first native leaflet of the cardiac valve during systole and (b) displace at least a portion of a second native leaflet of the cardiac valve, wherein the fixation member extends from the coaptation member, and wherein the at least one connector is positioned opposite the coaptation member relative to a vertical axis of the valve repair device.

10. The delivery system of claim 8, further comprising a delivery catheter, wherein the implant shaft extends through the delivery catheter, and wherein the hub assembly is configured to be releasably coupled to the at least one connector when the fixation member is at least partially expanded distal of the delivery catheter.

11. The delivery system of 10 wherein the valve repair device extends along a vertical axis, and wherein the hub assembly is configured to be releasably coupled to the at least one connector offset from the vertical axis when the fixation member is at least partially expanded distal of the delivery catheter.

12. A delivery system for endovascularly implanting a valve repair device at a cardiac valve, the delivery system comprising:
a clip cable configured to be releasably coupled to a clip assembly of the valve repair device;
a cinching line configured to extend at least partially around an expandable fixation member of the valve repair device and to cinch the fixation member to a compressed position; and
a handle having an actuator operably coupled to the clip cable and the cinching line, wherein the actuator is actuatable to simultaneously (a) reduce a tension in the cinching line to permit the fixation member to expand to an expanded position and (b) reduce a tension in the clip cable.

13. The delivery system of claim 12 wherein the handle further comprises a clip cable mount assembly operably coupled to the clip cable and a cinch mount assembly operably coupled to the cinching line, wherein the actuator is actuatable to simultaneously move the clip cable mount assembly and the cinch mount assembly in a same direction to reduce the tension in the cinching line and reduce the tension in the clip cable.

14. The delivery system of claim 13 wherein the actuator is actuatable to simultaneously move the clip cable mount assembly at a first rate and the cinch mount assembly at a second rate different than the first rate.

15. The delivery system of claim 14 wherein the second rate is between about 2.5-3.5 times faster than the first rate.

16. The delivery system of claim 13 wherein the clip cable mount assembly is movably mounted to a first lead screw, wherein the cinch mount assembly is movably mounted to a second lead screw, and wherein the actuator is rotatable to simultaneously (a) rotate the first lead screw to movably the clip cable mount assembly therealong to reduce the tension in the clip cable and (b) rotate the second lead screw to movably the cinch mount assembly therealong to reduce the tension in the cinching line.

17. The delivery system of claim 16 wherein the actuator is rotatable to simultaneously rotate the first lead screw at a first rate and the second lead screw at a second rate different than the first rate.

18. The delivery system of claim 17 wherein the second rate is between about 2.5-3.5 times faster than the first rate.

19. A handle of a delivery system for endovascularly implanting a valve repair device at a cardiac valve, the handle comprising:
a housing;
a first leadscrew carried by the housing;
a second leadscrew carried by the housing;
a cinch mount assembly operably coupled to the first leadscrew and configured to be coupled to a cinching line of the delivery system, wherein the cinching line is configured to extend at least partially around the valve repair device and to cinch the valve repair device to a compressed position;
a clip cable mount assembly operably coupled to the second leadscrew and configured to be coupled to a clip cable of the delivery system, wherein the clip cable is configured to be releasably coupled to a clip assembly of the valve repair device;
an actuator; and
a gear assembly operably coupling the actuator to the first and second leadscrews, wherein the actuator is actuatable to drive the gear assembly to simultaneously drive (a) the cinch mount assembly axially along the first leadscrew at a first rate and (b) the clip cable mount assembly axially along the second leadscrew at a second rate different than the first rate.

20. The handle of claim 19 wherein the second rate is between about 2.5-3.5 times faster than the first rate.

21. A delivery system for endovascularly implanting a valve repair device at a cardiac valve, the delivery system comprising:
a clip cable configured to be releasably coupled to a clip assembly of the valve repair device; and
a handle having a first actuator and a second actuator operably coupled to the clip cable, wherein the first actuator is actuatable to drive the clip cable axially relative to the handle, and wherein the second actuator is actuatable to rotate the clip cable to open and close the clip assembly.

22. The delivery system of claim 21 wherein the first actuator is actuatable to drive the clip cable axially relative to the handle to change a level of tension in the clip cable.

23. The delivery system of claim 21 wherein the handle further comprises a sleeve and an elongate member at least partially positioned within the sleeve, wherein the clip cable has a proximal portion fixedly coupled to the elongate member, wherein the first actuator is actuatable to move the elongate member at least partially through the sleeve, and wherein the second actuator is rotatable to rotate the sleeve to rotate the elongate member to rotate the clip cable to open and close the clip assembly.

24. The delivery system of claim 23 wherein the handle further comprises:
a leadscrew;
a clip cable carriage movably mounted to the leadscrew; and
at least one spring operably mounted between the clip cable carriage and the elongate member, wherein the first actuator is actuatable to move the clip cable carriage along the leadscrew, and wherein the at least one spring is configured to transmit at least a portion of the movement of the clip cable carriage to the elongate member such that the movement of the clip cable carriage moves the elongate member to move the clip cable axially relative to the handle.

25. The delivery system of claim 24 wherein the at least one spring comprises an extension spring.

26. The delivery system of claim 24 wherein the clip cable carriage further comprises at least one extension portion configured to bow the at least one spring outward away from a longitudinal axis of the handle.

27. The delivery system of claim 24 wherein the at least one spring is configured to transmit less than all of the movement of the clip cable carriage to the elongate member when the first actuator is actuated to move the clip cable carriage along the leadscrew at above a predetermined rate.

28. A handle of a delivery system for endovascularly implanting a valve repair device at a cardiac valve, the handle comprising:
a housing;
a leadscrew positioned within the housing;
a clip cable mount assembly operably coupled to the leadscrew, wherein the clip cable mount assembly is configured to be coupled to a clip cable of the delivery system, and wherein the clip cable is configured to be releasably coupled to a clip assembly of the valve repair device;
a sleeve positioned around a portion of the clip cable mount assembly;
a first actuator operably coupled to the clip cable mount assembly, wherein the first actuator is actuatable to drive the clip cable mount assembly and the clip cable axially along the leadscrew, and wherein the portion of the clip cable mount assembly is configured to telescope within the sleeve as the clip cable mount assembly moves axially along the leadscrew; and
a second actuator operably coupled to the sleeve, wherein the second actuator is actuatable to rotate the sleeve such that the clip cable mount assembly rotates the clip cable to open and close the clip assembly.

29. The handle of claim 28 wherein the sleeve includes an inner surface, wherein the portion of the clip cable mount assembly has an outer surface, and wherein the inner surface and the outer surface have a same cross-sectional shape.

30. The handle of claim 28 wherein the sleeve has at least one slot, wherein the portion of the clip cable mount assembly has an outer surface including at least one projection extending therefrom, and wherein the projection extends through the slot.

31. A method of implanting a valve repair device at a cardiac valve, the method comprising:
endovascularly delivering a distal portion of a delivery catheter to a chamber of a heart;
unsheathing at least a portion of the valve repair device from the delivery catheter while in the chamber of the heart;
advancing the valve repair device to a target position extending across the cardiac valve such that a fixation member of the valve repair device is positioned at a first side of the cardiac valve upstream of a native valve anulus of the cardiac valve and a coaptation member of the valve repair device is positioned at least partially at a second side of the cardiac valve proximate to native valve leaflets of the cardiac valve;
rotating, in a first direction, a clip cable coupled to a clip assembly extending from the coaptation member to open the clip assembly;
rotating, in a second direction, the clip cable to close the clip assembly to capture at least a portion of one of the native valve leaflets between the clip assembly and the coaptation member;
reducing a tension in a cinching line extending at least partially around the fixation member to allow the fixation member to at least partially expand; and
while reducing the tensioning in the cinching line, reducing a tension in the clip cable.

32. The method of claim 31 wherein endovascularly delivering the distal portion of the delivery catheter to the chamber of the heart comprises advancing the distal portion with a portion of the coaptation member extending distally from the distal portion of the delivery catheter.

33. The method of claim 32 wherein the portion of the coaptation member is atraumatic.

34. The method of claim 31 wherein reducing the tension in the cinching line further comprises permitting an implant shaft releasably coupled to the fixation member to move away from a vertical axis of the valve repair device.

35. The method of claim 34 wherein the fixation member extends in a superior direction from the coaptation member, wherein the fixation member includes a superior end portion and an inferior end portion, wherein the valve repair device further comprises at least one connector extending radially inward from the fixation member from between the superior end portion and the inferior end portion, and wherein the implant shaft has a hub assembly configured to be releasably coupled to the at least one connector.

36. The method of claim 35 wherein the method further comprises, after reducing the tension in the cinching line, actuating a drive shaft operably coupled to the hub assembly to release the at least one connector from the hub assembly to decouple the valve repair device from the implant shaft.

37. The method of claim 31 wherein the cardiac valve is a native mitral valve.
